SEQUENCE LISTING (5 sequences)

SEQ ID NO: 1 - DNA, 19 bp, Artificial Sequence
  Synthetic Oligonucleotide Primer
cggggttcagtacatttat

SEQ ID NO: 2 - DNA, 21 bp, Artificial Sequence
  Synthetic Oligonucleotide Primer
gatcttaggaccacccaactg

SEQ ID NO: 3 - DNA, 28 bp, Artificial Sequence
  Synthetic Oligonucleotide Primer
aattcctgttggtgtcatagctgtggag

SEQ ID NO: 4 - DNA, 28 bp, Artificial Sequence
  Synthetic Oligonucleotide Primer
ttcagatttatcaactctgggtcaagcc

SEQ ID NO: 5 - DNA, 1112 bp, Artificial Sequence
  Amplified PCR product
ggcgattgggccgaagtcgcatgctcccggccgccatggcggccgcgggaattcgatacccctttttcagtacatttatctgactgaagaagattatgctcgtattagctcttctgttatagcacataagctacagctggacagcggtgaaattaggtggattattgactctgttgtgggcaaggaggatgggcttggtgttgagaatttacatggaagtgctgctattgccagtgcttattctagggcatacgaggagacatttacacttacgttcgtgactgggcggactgtaggaataggagcttatcttgcacgacttggtatacggtgcatacagcgtcttgaccagcccattattttaacagggttttctgccctgaacaagcttcttgggcgtgaagtttacagctcccacatgcagttgggtggtcctaagatcatggcgacgaatggtgttgtccacctcactgtttcagatgatcttgaaggtgtatccagtatattgaggtggctcagctatgttcctgccaacattggtggacctcttcctattacaaaacctttggacccaccggacagacctgttgcgtacatccctgagaacacatgcgatccacgtgcagccatccgtggtgtagatgacagccaagggcaatggttgggtggtatgtttgacaaagacagctttgtggagacatttgaaggatgggcgaaaacagttgtcactggcagggcatagcttggaggaattcctgtgggtgtcatagctgtggagacacagaacatgatgcagctcatccctgctgatccaggccagcttgattctcatgagcgatctgttcctcgggctgaacaagtgtggttcccagattctgcaaccaagactgctcaagcattgttggacttcaaccgtgaaggattgcctctgttcatccttgctaactggagaggtttctctggtggacaaagagatctctttgaaggaattcttcaggctgggtcaacaattgttgagaaccttaggacgtacaatcaacctgcgtttgtctacattcctatggctggagagctgcgtggaggagcttgggttgtggttgatagcaaaataa

(19) United States
(12) Patent Application Publication
(10) Pub. No.: US 2010/0048405 A1
(43) Pub. Date: Feb. 25, 2010

(54) DEVELOPMENT OF HERBICIDE-RESISTANT GRASS SPECIES

(75) Inventors: Paul L. Raymer, Milner, GA (US); Douglas Heckart, Athens, GA (US); Wayne Allen Parrott, Athens, GA (US)

Correspondence Address: DAVIS WRIGHT TREMAINE LLP - San Francisco, 505 MONTGOMERY STREET, SUITE 800, SAN FRANCISCO, CA, 94111, US

(73) Assignee: University of Georgia Research Foundation, Athens, GA (US)

(21) Appl. No.: 12/488,452

(22) Filed: Jun. 19, 2009

Related U.S. Application Data
(60) Provisional application No. 61/074,381, filed on Jun. 20, 2008.
(60) Provisional application No. 61/150,459, filed on Feb. 6, 2009.
(60) Provisional application No. 61/172,427, filed on Apr. 24, 2009.

(51) Int. Cl. A01N 35/10; A01H 5/00; A01H 1/06; C12Q 1/02; C12N 5/04; C07H 21/00; A01P 13/00

(52) U.S. Cl. 504/343; 800/300; 800/267; 800/265; 800/264; 800/263; 435/29; 435/410; 536/23.1

(57) ABSTRACT

The invention relates to a selected and cultured ACCase inhibitor herbicide-resistant plant-resistant plant from the group Panicodae, or tissue, seed, or progeny thereof, and methods of selecting the same. The invention also relates to methods for controlling weeds in the vicinity of an ACCase inhibitor herbicide-resistant plant.

### CROSS-REFERENCE TO RELATED APPLICATIONS

This application claims priority from U.S. Provisional Application Ser. No. 61/074,381, filed on Jun. 20, 2008, U.S. Provisional Application Ser. No. 61/150,459, filed on Feb. 6, 2009, and U.S. Provisional Application Ser. No. 61/172,427, filed on Apr. 24, 2009, each of which is incorporated herein by reference in its entirety.

### FIELD

The invention disclosed herein generally relates to grasses with resistance to selective grass herbicides and methods to develop the same.

### BACKGROUND

Seashore paspalum (Paspalum vaginatum) is a warm-season turfgrass that is generally adapted to dune environments. Favorable attributes of seashore paspalum include its tolerance to salt, water logging, and drought. These characteristics make paspalum a premium turfgrass candidate for venues where any or all of these environmental problems could be an issue. For example, golf course architects recommend seashore paspalum for new courses in tropical or sub-tropical coastal areas where salt or water quality can affect turfgrass growth and maintenance. In addition, many existing golf courses have replaced bermudagrass (Cynodon dactylon) with paspalum. Compared to bermudagrass, paspalum requires less nitrogen and is more tolerant of irrigation with brackish or poor quality water, which reduces management costs and improves irrigation flexibility.

A main limitation to replacing bermudagrass with paspalum is bermudagrass re-establishment. Bermudagrass is highly competitive and difficult to eradicate once established. Bermudagrass and other weedy grasses can greatly reduce the aesthetic value and quality of the paspalum turf. Accordingly, it is desired to control or limit bermudagrass or weedy grass growth in paspalum-populated areas. To control the growth of weedy grasses in paspalum-populated turfgrass areas, the development of paspalum turfgrass with resistance to selective grass herbicides is desired. Past approaches in development of herbicide-resistant turfgrass include the use of genetic engineering approaches. However, plants produced by genetic engineering approaches may be difficult to commercialize due to governmental regulations and restrictions regarding the use of genetically modified plants. Accordingly, embodiments of the invention include the development of turfgrass cultivars with non-transgenic resistance to herbicides, as well as cultivars with transgenic resistance.

### SUMMARY

Embodiments of the invention relate to a selected and cultured ACCase inhibitor herbicide-resistant plant-resistant plant from the group Panicodae, or tissue, seed, or progeny thereof. In some embodiments, the ACCase inhibitor herbicide-resistant plant is regenerated from an herbicide-resistant undifferentiated cell that has undergone a selection method, wherein the selection method includes: providing a callus of undifferentiated cells of a plant from the group Panicodae, contacting the callus with at least one herbicide in an amount sufficient to retard growth or kill the callus, selecting at least one resistant cell based upon a differential effect of the herbicide, and regenerating a viable whole plant of the variety from the at least one resistant cell. In some embodiments, the plant is a non-transgenic plant.

In some embodiments of the invention, the ACCase inhibitor herbicide-resistant plant is a member of tribe Paniceae. In some embodiments, the ACCase inhibitor herbicide-resistant plant is one selected from the group of: Axonopus (carpetgrass), Digiteria (crabgrass), Echinochloa, Panicum, Paspalum (Bahiagrass), Pennisetum, Setaria and Stenotaphrum (St. Augustine grass). In some embodiments, the ACCase inhibitor herbicide-resistant plant is one selected from the group of: seashore paspalum (P. vaginatum), bent grass, tall fescue grass, Zoysiagrass, bermudagrass (Cynodon spp), Kentucky Bluegrass, Texas Bluegrass, Perennial ryegrass, buffalograss (Buchloe dactyloides), centipedegrass (Eremochloa ophiuroides) and St. Augustine grass (Stenotaphrum secundatum), Carpetgrass (Axonopus spp.) and Bahiagrass (Paspalum notatum).

In some embodiments of the invention, the ACCase inhibitor herbicide-resistant plant is resistant to an acetyl coenzyme A carboxylase (ACCase) inhibitor. In some embodiments, the ACCase inhibitor herbicide-resistant plant is resistant to a cyclohexanedione herbicide, an aryloxyphenoxy proprionate herbicide, a phenylpyrazoline herbicide, or mixtures thereof. In some embodiments, the ACCase inhibitor herbicide-resistant plant is resistant to at least one herbicide selected from the group of: alloxydim, butroxydim, cloproxydim, profoxydim, sethoxydim, clefoxydim, clethodim, cycloxydim, tepraloxydim, tralkoxydim, chloraizfop, clodinafop, clofop, cyhalofop, diclofop, fenoxaprop, fenthiaprop, fluazafop-butyl, fluazifop, haloxyfop, isoxapyrifop, metamifop, propaquizafop, quizalofop, trifop and pinoxaden.

In some embodiments of the invention, the herbicide resistance of the ACCase inhibitor herbicide-resistant plant is conferred by a mutation at least one amino acid position of ACCase gene selected from the group of: 1756, 1781, 1999, 2027, 2041, 2078, 2099 and 2096. In some embodiments, the herbicide resistance is conferred by an isoleucine to leucine mutation at amino acid position 1781.

Embodiments of the invention also relate to a progeny of an ACCase inhibitor herbicide-resistant plant plant as described in any of the foregoing paragraphs. In some embodiments, the progeny is a result of sexual reproduction of the ACCase inhibitor herbicide-resistant plant parent. In some embodiments, the progeny is a result of asexual reproduction of the ACCase inhibitor herbicide-resistant plant parent.

Embodiments of the invention are also directed to a seed of an ACCase inhibitor herbicide-resistant plant as described in any of the foregoing paragraphs, or a progeny thereof.

Embodiments of the invention relate to sod comprising an ACCase inhibitor herbicide-resistant plant of as described in any of the foregoing paragraphs, or a progeny or seed thereof. Embodiments of the invention are also directed a turfgrass nursery plot comprising an ACCase inhibitor herbicide-resistant plant as described in any of the foregoing paragraphs, or a progeny or seed thereof. In embodiments of the invention, a commercial lawn, golfcourse, or field comprising an ACCase inhibitor herbicide-resistant plant as described in any of the foregoing paragraphs, or a progeny or seed thereof, is provided.

Embodiments of the invention also relate to a method of identifying a herbicide-resistant plant from the group Panicodae, including: providing a callus of undifferentiated cells of a plant from the group Panicodae, contacting the callus with at least one herbicide in an amount sufficient to retard growth or kill the callus, selecting at least one resistant cell based upon a differential effect of the herbicide, and regenerating a viable whole plant of the variety from the at least one resistant cell, wherein the regenerated plant is resistant to the at least one herbicide. In some embodiments, the method further includes expanding the at least one resistant cell into a plurality of undifferentiated cells. In some embodiments, the callus of undifferentiated cells is provided from a non-transgenic plant.

In some embodiments of the invention, the plant provided in the method is one selected from the tribe Paniceae. In some embodiments, the plant is one selected from the group of: Axonopus (carpetgrass), Digiteria (crabgrass), Echinochloa, Panicum, Paspalum (Bahiagrass), Pennisetum, Setaria and Stenotaphrum (St. Augustine grass). In some embodiments, the plant is one selected from the group of: seashore paspalum (P. vaginatum), bentgrass (Agrostis spp), tall fescue, Zoysiagrass, bermudagrass (Cynodon spp), Kentucky Bluegrass, Texas Bluegrass, Perennial ryegrass, buffalograss (Buchloe dactyloides), centipedegrass (Eremochloa ophiuroides) and St. Augustine grass (Stenotaphrum secundatum), Carpetgrass (Axonopus spp.) and Bahiagrass (Paspalum notatum).

In some embodiments of the invention, the at least one herbicide used in the method is an acetyl coenzyme A carboxylase (ACCase) inhibitor. In some embodiments, the at least one herbicide is selected from the group of: alloxydim, butroxydim, cloproxydim, profoxydim, sethoxydim, clefoxydim, clethodim, cycloxydim, tepraloxydim, tralkoxydim, chloraizfop, clodinafop, clofop, cyhalofop, diclofop, fenoxaprop, fenthiaprop, fluazafop-butyl, fluazifop, haloxyfop, isoxapyrifop, metamifop, propaquizafop, quizalofop, trifop and pinoxaden.

In some embodiments of the invention, the herbicide resistance of the plant is conferred by a mutation at least one amino acid position of the ACCase gene selected from the group of: 1756, 1781, 1999, 2027, 2041, 2078, 2099 and 2096. In some embodiments, the herbicide resistance is conferred by an isoleucine to leucine mutation at amino acid position 1781.

Embodiments of the invention are also directed to a tissue culture of regenerable cells of an herbicide-resistant plant identified by the methods as described in the foregoing paragraphs.

In embodiments of the invention, a method for controlling weeds in the vicinity of a herbicide-resistant plant is provided, wherein the herbicide-resistant plant is identified by the methods described in the foregoing paragraphs, the method including: contacting at least one herbicide to the weeds and to the herbicide-resistant plant, wherein the at least one herbicide is contacted to the weeds and to the plant at a rate sufficient to inhibit growth of a non-selected plant of the same species or sufficient to inhibit growth of the weeds. In some embodiments, the herbicide-resistant plant is resistant to an acetyl coenzyme A carboxylase (ACCase) inhibitor. In some embodiments, the method includes contacting the herbicide directly to the herbicide-resistant plant. In some embodiments, the method includes contacting the herbicide to a growth medium in which the herbicide-resistant plant is located.

In some embodiments, the herbicide-resistant plant is resistant to a cyclohexanedione herbicide, an aryloxyphenoxy proprionate herbicide, a phenylpyrazoline herbicide, or mixtures thereof. In some embodiments, the herbicide-resistant plant is a non-transgenic plant.

In some embodiments of the invention, the herbicide resistance in the plant is conferred by a mutation at least one amino acid position of the ACCase gene selected from the group of: 1756, 1781, 1999, 2027, 2041, 2078, 2099 and 2096. In some embodiments, the herbicide resistance is conferred by an isoleucine to leucine mutation at amino acid position 1781 of the ACCase gene.

In some embodiments, the at least one herbicide used in the method is selected from the group of: alloxydim, butroxydim, cloproxydim, profoxydim, sethoxydim, clefoxydim, clethodim, cycloxydim, tepraloxydim, tralkoxydim, chloraizfop, clodinafop, clofop, cyhalofop, diclofop, fenoxaprop, fenthiaprop, fluazafop-butyl, fluazifop, haloxyfop, isoxapyrifop, metamifop, propaquizafop, quizalofop, trifop and pinoxaden.

Embodiments of the invention are directed to a seashore paspalum-specific DNA marker deposited as ATCC Deposit No. ______, or a fragment thereof, that is capable of identifying herbicide-resistant grass cultivars. In some embodiments, the seashore-paspalum-specific DNA marker comprises SEQ ID NO: 5, or a fragment thereof.

Embodiments of the invention also relate to a method of identifying a herbicide-resistant plant, including: obtaining a genetic sample of a plant, and assaying the sample for the presence or absence of a mutation at position 1781 of the ACCase gene, wherein the presence of a mutation at position 1781 is indicative of herbicide-resistance in the plant. Also contemplated are uses of the marker at position 1781 of the ACCase in a method of identifying an herbicide-resistant plant.

Embodiments of the invention are drawn to a method of marker-assisted breeding, including the steps of: identifying a feature of interest for breeding and selection, wherein the feature is in linkage with an ACCase gene, providing a first plant carrying an ACCase sequence variant capable of conferring upon the plant resistance to an ACCase-inhibitor herbicide, wherein the plant further comprises the feature of interest, breeding the first plant with a second plant, identifying progeny of the breeding step as having the ACCase sequence variant; and selecting progeny likely to have the feature of interest based upon the identifying step. In some embodiments, the feature is selected from: a trait or, a gene. In some embodiments, the trait is at least one selected from the group consisting of: herbicide tolerance, disease resistance, insect of pest resistance, altered fatty acid, protein or carbohydrate metabolism, increased growth rates, enhanced stress tolerance, preferred maturity, enhanced organoleptic properties, altered morphological characteristics, sterility, other agronomic traits, traits for industrial uses, or traits for improved consumer appeal.

In some embodiments of the invention, the ACCase sequence variant included within the method includes a variation at least one of position: 1756, 1781, 1999, 2027, 2041, 2078, 2099 and 2096. In some embodiments, the herbicide to which the plant is resistant is at least one selected from the group of: alloxydim, butroxydim, cloproxydim, profoxydim, sethoxydim, clefoxydim, clethodim, cycloxydim, tepraloxydim, tralkoxydim, chloraizfop, clodinafop, clofop, cyhalofop, diclofop, fenoxaprop, fenthiaprop, fluazafop-butyl, fluazifop, haloxyfop, isoxapyrifop, metamifop, propaquizafop, quizalofop, trifop and pinoxaden.

In some embodiments, the identifying step included within the method includes a process selected from: molecular detection of the sequence variant, observation of resistance to an ACCase inhibitor, and selection by application of an ACCase inhibitor.

Embodiments of the invention relate to a transgenic plant, transformed with a segment of DNA comprising at least 250 bases derived from the sequence of ATCC Deposit No. ______, and progeny plants of the same. In some embodiments, the progeny plant is selected from: a backcross progeny, a hybrid, a clonal progeny, and a sib-mated progeny. In some embodiments, the segment of DNA comprises at least 250 bases derived from SEQ ID NO: 5.

Embodiments of the invention are also directed to a transformed cell containing a segment of DNA comprising at least 250 bases derived from the sequence of ATCC Deposit No. ______. In some embodiments, the segment of DNA comprises at least 250 bases derived from SEQ ID NO: 5.

In embodiments of the invention, a method of identifying a mutation at position 1781 of the ACCase gene in a cell is provided, the method including obtaining a genetic sample from a cell, selectively amplifying a DNA fragment by using SV384F primer and SV348R primer in an amplification step, and sequencing the DNA fragment to determine the presence of absence of a mutation at position 1781 of the ACCase gene, wherein the presence of a mutation in the DNA fragment is indicative of the presence of the mutation at position 1781 in the cell.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 is a diagram of the fatty acid biosynthesis pathway in plants.

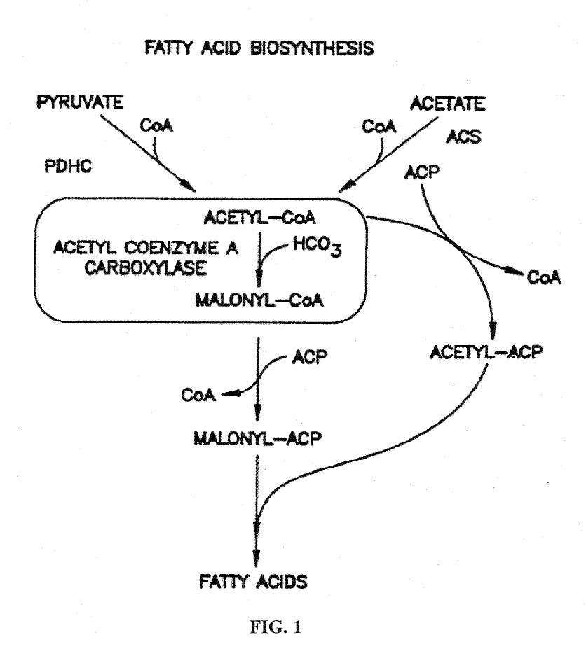

FIG. 2 is an illustration of an embodiment of a herbicide selection protocol for selecting non-transgenic herbicide-resistant plants as disclosed herein.

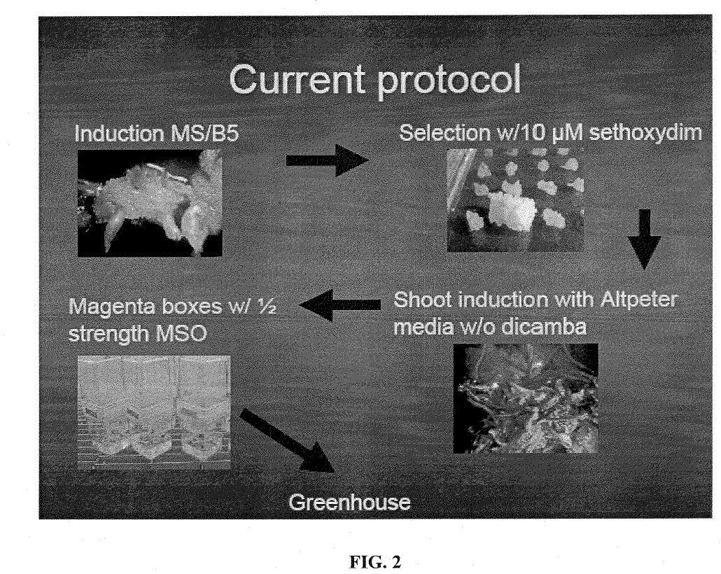

FIG. 3 is a graph illustrating a sethoxydim dose-response curve for seashore paspalum (Paspalum vaginatum).

FIG. 4 is a photograph of a sethoxydim-resistant callus of seashore paspalum growing on callus induction medium containing sethoxydim.

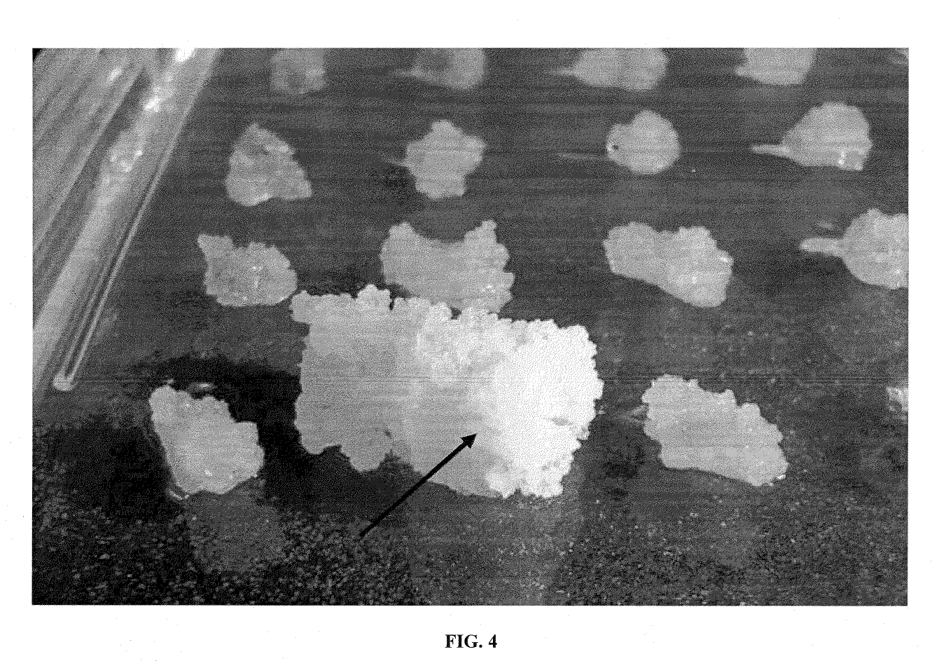

FIG. 5 is a series of chromatographs illustrating the amino acid mutation at position 1781 of the ACCase gene in an herbicide-resistant seashore paspalum plant selected as disclosed herein.

FIG. 6 is a photograph illustrating the response of control plants and herbicide-resistant plants, selected as disclosed herein, to Segment™ sethoxydim at 7 days after treatment (DAT).

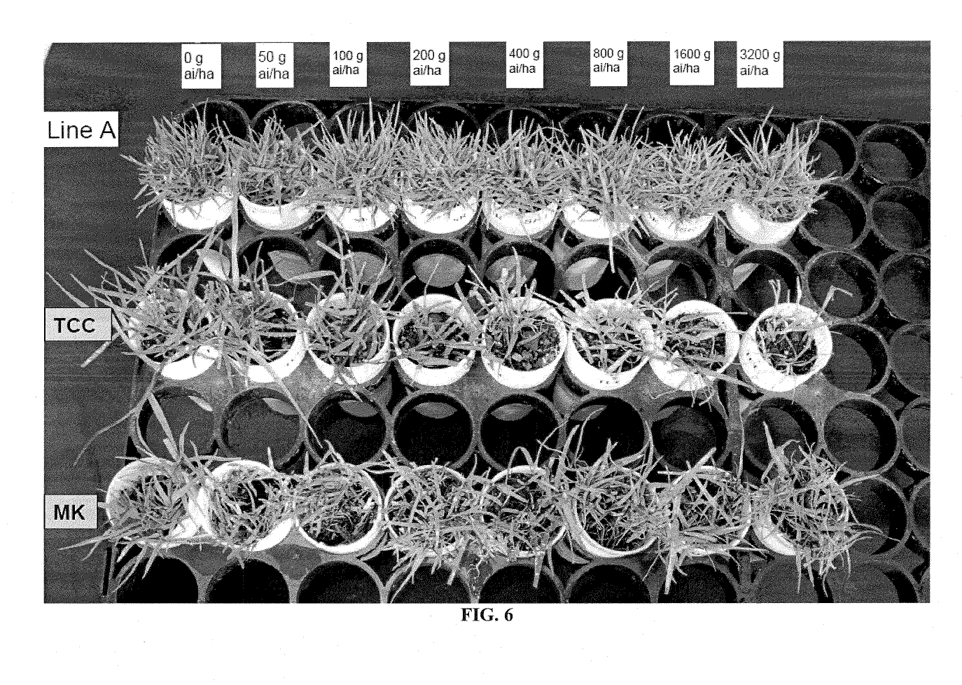

FIG. 7 is graph that illustrates injury to control plants and herbicide-resistant plants, selected as disclosed herein, by Segment™ sethoxydim at 7 days after treatment (DAT).

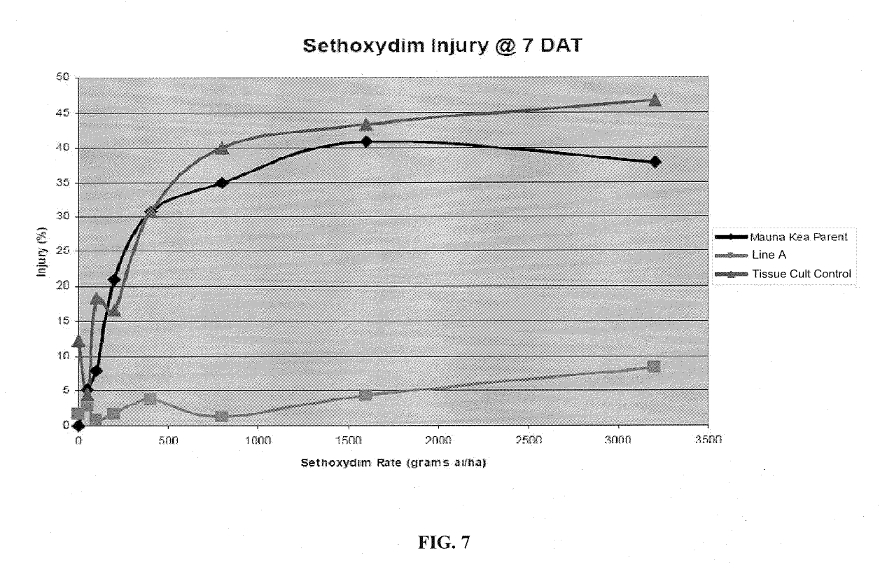

FIG. 8 is a photograph illustrating the response of control plants and herbicide-resistant plants, selected as disclosed herein, to Segment™ sethoxydim at 14 days after treatment (DAT).

FIG. 9 is graph that illustrates injury to control plants and herbicide-resistant plants, selected as disclosed herein. by Segment™ sethoxydim at 14 days after treatment (DAT).

FIG. 10 is a photograph illustrating the response of control plants and herbicide-resistant plants, selected as disclosed herein, to Segment™ sethoxydim at 21 days after treatment (DAT).

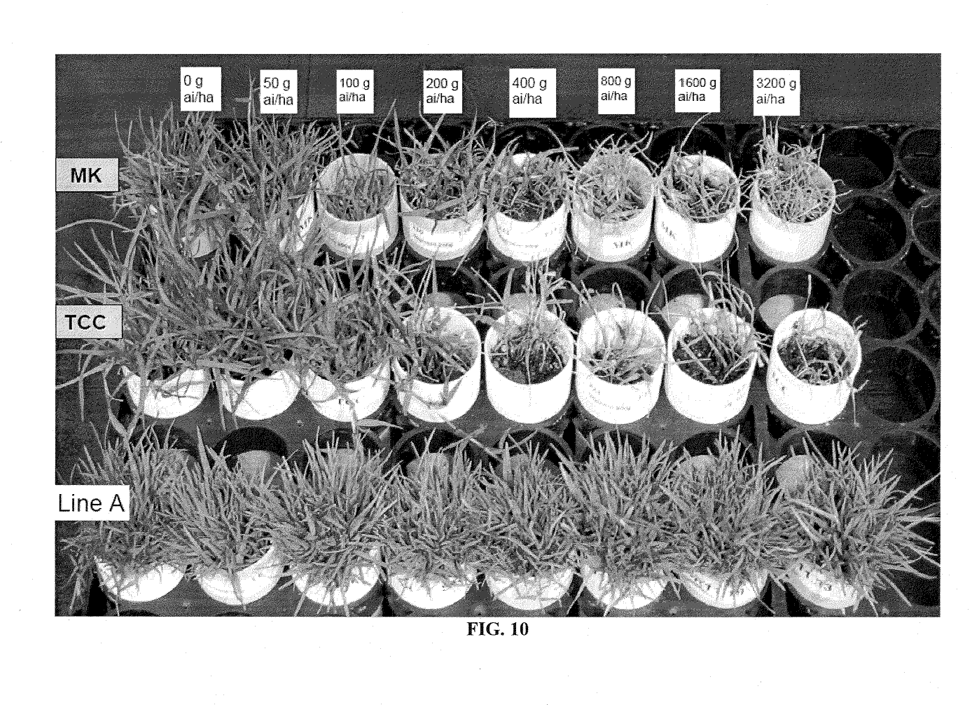

FIG. 11 is graph that illustrates injury to control plants and herbicide-resistant plants, selected as disclosed herein, by Segment™ sethoxydim at 21 days after treatment (DAT).

FIG. 12 is a graph that illustrates the mean dry weight of control plants and herbicide-resistant plants, selected as disclosed herein, after treatment with Segment™ sethoxydim at 42 days after treatment (DAT).

FIG. 13 is a graph that illustrates injury to control plants and herbicide-resistant plants, selected as disclosed herein, by Poast™ sethoxydim at 21 days after treatment (DAT).

FIG. 14 is a graph that illustrates injury to control plants and herbicide-resistant plants, selected as disclosed herein, by Fusilade II™ fluazifop-p-butyl herbicide at 21 days after treatment (DAT).

FIG. 15 is a graph that illustrates injury to control plants and herbicide-resistant plants, selected as disclosed herein, by Acclaim Extra™ II fenoxaprop-p-butyl herbicide at 21 days after treatment (DAT).

FIG. 16 is an illustration of an embodiment of callus production obtained from the intercalary meristem of a plant.

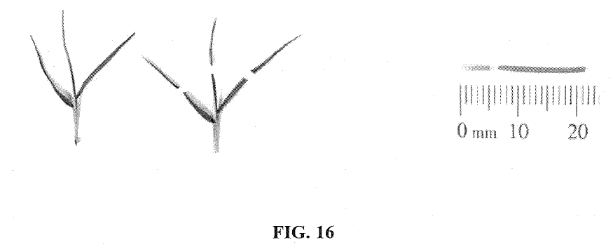

### DETAILED DESCRIPTION

Resistance to selective grass herbicides can provide a highly effective means of controlling weedy grasses in various turf grass species. Genetic engineering approaches have been proposed for the development of herbicide-resistant plants, however, these can be difficult to commercialize due to governmental regulations and restrictions regarding the use of genetically modified plants. In contrast, environmental release of plants with herbicide resistance derived by non-transgenic means is not currently subjected to strict governmental regulation. Accordingly, embodiments of the invention relate to methods of screening and selecting herbicide-resistant turf grass plants, including methods that are effective without transgenesis.

### Definitions

Unless otherwise noted, terms are to be understood according to conventional usage by those of ordinary skill in the relevant art.

As used herein, the term “explant” refers to a plant tissue that includes meristematic tissue. It can also refer to plant tissues that include, without limitation, one or more embryos, cotyledons, hypocotyls, leaf bases, mesocotyls, plumules, protoplasts and embryonic axes.

As used herein, the term “callus” refers to an undifferentiated plant cell mass that can be grown or maintained in a culture medium to produce genetically identical cells.

As used herein, the term “herbicide-resistant” or “herbicide-tolerant,” including any of their variations, refers to the ability of a plant to recover from, survive and/or thrive after contact with an herbicide in an amount that is sufficient to cause retardation of growth or death of a non-resistant plant of the same species. Typically, amounts of herbicide sufficient to cause growth or death of a non-resistant plant ranges from about 2 μM to about 100 μM of herbicide concentration. In some embodiments, a sufficient amount of herbicide ranges from about 5 μM to about 50 μM of herbicide concentration, from about 8 μM to about 30 μM of herbicide concentration, or from about 10 μM to about 25 μM of herbicide concentration. Alternatively, amounts of herbicide sufficient to cause growth or death of a non-resistant plant ranges from about 25 grams active ingredient per hectare (g ai ha⁻¹) to about 6500 g ai ha⁻¹ of herbicide application. In some embodiments, a sufficient amount of herbicide ranges from about 50 g ai ha⁻¹ to about 5000 g ai ha⁻¹ of herbicide application, about 75 g ai ha⁻¹ to about 2500 g ai ha⁻¹ of herbicide application, about 100 g ai ha⁻¹ to about 1500 g ai ha⁻¹ of herbicide application, or about 250 g ai ha⁻¹ to about 1000 g ai ha⁻¹ of herbicide application.

As used herein, the term “marker-assisted selection” refers to to the process of selecting a desired trait or desired traits in a plant or plants by detecting one or more markers in linkage with the desired trait. Such markers can be phenotypic markers such as, for example, resistance to an herbicide or antibiotic. Likewise, such markers can be molecular markers such as, for example, one or more polymorphisms (as described below), DNA or RNA enzymes, or other sequences that are easily detectable.

A polynucleotide “exogenous” to an individual plant is a polynucleotide which is introduced into the plant by any means other than by a sexual cross. Examples of means by which this can be accomplished are described below, and include transformation, biolistic methods, electroporation, and the like. Such a plant containing the exogenous nucleic acid is referred to here as R₀ (for plants regenerated from transformed cells in vitro) generation transgenic plant. R₀ can also refer to any other regenerated plant whether transgenic or not.

As used herein, the term “transgenic” describes a non-naturally occurring plant that contains a genome modified by man, wherein the plant includes in its genome an exogenous nucleic acid molecule, which can be derived from the same or a different species, including non-plant species. The exogenous nucleic acid molecule can be a gene regulatory element such as a promoter, enhancer, or other regulatory element, or can contain a coding sequence, which can be linked to a native or heterologous gene regulatory element. Transgenic plants that arise from sexual cross or by selfing are descendants of such a plant.

As used herein, “polymorphism” means the presence of one or more variations of a nucleic acid sequence at one or more loci in a population of one or more individuals. The variation can comprise, but is not limited to, one or more base changes, the insertion of one or more nucleotides, or the deletion of one or more nucleotides. A polymorphism includes a single nucleotide polymorphism (SNP), a simple sequence repeat (SSR), indels (insertions and deletions), a restriction fragment length polymorphism, a haplotype, and a tag SNP. In addition, a polymorphism can include a genetic marker, a gene, a DNA-derived sequence, a RNA-derived sequence, a promoter, a 5′ untranslated region of a gene, a 3′ untranslated region of a gene, microRNA, siRNA, a quantitative trait locus (QTL), a satellite marker, a transgene, mRNA, ds mRNA, a transcriptional profile, or a methylation pattern. A polymorphism can arise from random processes in nucleic acid replication, through mutagenesis, as a result of mobile genomic elements, from copy number variation and during the process of meiosis, such as unequal crossing over, genome duplication and chromosome breaks and fusions. The variation can be commonly found or can exist at low frequency within a population, the former having greater utility in general plant breeding and the later can be associated with rare but important phenotypic variation.

As used herein, a “marker” refers to a polymorphic nucleic acid sequence or nucleic acid feature. In a broader aspect, a “marker” can be a detectable characteristic that can be used to discriminate between heritable differences between organisms. Examples of such characteristics can include genetic markers, protein composition, protein levels, oil composition, oil levels, carbohydrate composition, carbohydrate levels, fatty acid composition, fatty acid levels, amino acid composition, amino acid levels, biopolymers, pharmaceuticals, starch composition, starch levels, fermentable starch, fermentation yield, fermentation efficiency, energy yield, secondary compounds, metabolites, morphological characteristics, and agronomic characteristics.

As used herein, a “marker assay” refers to a method for detecting a polymorphism at a particular locus using a particular method, e.g. measurement of at least one phenotype (such as seed color, flower color, or other visually detectable trait), restriction fragment length polymorphism (RFLP), single base extension, electrophoresis, sequence alignment, allelic specific oligonucleotide hybridization (ASO), random amplified polymorphic DNA (RAPD), microarray-based technologies, and nucleic acid sequencing technologies, etc.

As used herein, a “genotype” refers to the genetic component of the phenotype, and this can be indirectly characterized using markers or directly characterized by nucleic acid sequencing. Suitable markers include a phenotypic character, a metabolic profile, a genetic marker, or some other type of marker. A genotype can constitute an allele for at least one genetic marker locus or a haplotype for at least one haplotype window. In some embodiments, a genotype can represent a single locus, and in others it can represent a genome-wide set of loci. In some embodiments, the genotype can reflect the sequence of a portion of a chromosome, an entire chromosome, a portion of the genome, and the entire genome.

As used herein, “quantitative trait locus (QTL)” refers to a locus that controls to some degree numerically representable traits that are usually continuously distributed.

As used herein, a “nucleic acid sequence fragment” refers to a portion of a nucleotide sequence of a polynucleotide or a portion of an amino acid sequence of a polypeptide. Fragments of a nucleotide sequence can encode protein fragments that retain the biological activity of the native or corresponding full-length protein. Fragments of a nucleotide sequence can range from at least about 20 nucleotides, about 50 nucleotides, about 100 nucleotides, about 250 nucleotides and up to the full-length nucleotide sequence of genes or sequences encoding proteins as disclosed herein.

### Suitable Plants for Screening

Embodiments of the invention are directed to herbicide-resistant plants from the group Panicodae regenerated from an herbicide-resistant cell that has undergone a herbicide selection process as well as methods of identifying the same. The plant can be, for example, one selected from the group of: an Isachneae tribe, a Neurachneae tribe, an Arundinellaeae tribe, and a Paniceae tribe. In some embodiments, the plant can be any member of a genus selected from the list provided in Table A or Table B. An exemplary, non-exhaustive list of plants suitable for use in the invention include members of the paniceae tribe, such as: Carpetgrass, Crabgrass, Bahiagrass, St. Augustine grass and millets, including Foxtail (Setaria italical), Pearl (Pennisetum glaucum), and Proso (Panicum miliaceum; commonly referred to as “common” millet, broom corn millet, hog millet or white millet).

In some embodiments, the plant is a turfgrass species having commercial value in applications such as, for example, golf courses, athletic fields, commercial landscaping, commercial or home lawns, and pastures. Exemplary turfgrass species include, but are not limited to, seashore paspalum (Paspalum vaginatum), bahiagrass (Paspalum notatum), bermudagrass (Cynodon spp.), blue gramma grass, buffalograss (Buchloe dactyloides), carpetgrass (Axonopus spp.), centipedegrass (Eremochloa ophiuroides), kikuyugrass, sideoats grama, St. Augustine grass (Stenotaphrum secondatum), Zoysiagrass, annual bluegrass, annual ryegrass, Canada bluegrass, chewings fescue, colonial bentgrass, creeping bentgrass, crested wheatgrass, fairway wheatgrass, hard fescue, Kentucky bluegrass, Texas bluegrass, orchard grass, perennial ryegrass, red fescue, redtop, rough bluegrass, sheep fescue, smooth bromegrass, tall fescue, Timothygrass, velvet bentgrass, weeping alkaligrass, western wheatgrass, and the like.

TABLE A
Genus members (organized by tribe) of Group Panicodae

| Tribe: Isachneae | Tribe: Neurachneae | Tribe: Arundinelleae |
|---|---|---|
| Coelachne | Neurachne | Arundinella |
| Cyrtococcum | Paraneurachne | Chandrasekharania |
| Heteranthoecia | Thyridolepis | Danthoniopsis |
| Hubbardia | | Diandrostachya |
| Isachne | | Dilophotriche |
| Limnopoa | | Garnotia |
| Sphaerocaryum | | Gilgiochloa |
| | | Isalus |
| | | Jansenella |
| | | Loudetia |
| | | Loudetiopsis |
| | | Trichopteryx |
| | | Tristachya |
| | | Zonotriche |

TABLE B
Genus members of tribe Paniceae of Group Panicodae
Tribe: Paniceae

Achlaena
Acostia
Acritochaete
Acroceras
Alexfloydia
Alloteropsis
Amphicarpum
Ancistrachne
Anthaenantiopsis
Anthenantia
Anthephora
Arthragrostis
Arthropogon
Axonopus
Baptorhachis
Beckeropsis
Boivinella
Brachiaria
Calyptochloa
Camusiella
Cenchrus
Centrochloa
Chaetium
Chaetopoa
Chamaeraphis
Chasechloa
Chloachne
Chlorocalymma
Cleistochloa
Cliffordiochloa
Commelinidium
Cymbosetaria
Cyphochlaena
Dallwatsonia
Dichanthelium
Digitaria
Digitariopsis
Dimorphochloa
Dissochondrus
Eccoptocarpha
Echinochloa
Echinolaena
Entolasia
Eriochloa
Fasciculochloa
Gerritea
Holcolemma
Homolepis
Homopholis
Hydrothauma
Hygrochloa
Hylebates
Hymenachne
Ichnanthus
Ixophorus
Lasiacis
Lecomtella
Leptocoryphium
Leptoloma
Leucophrys
Louisiella
Megaloprotachne
Melinis
Mesosetum
Microcalamus
Mildbraediochloa
Odontelytrum
Ophiochloa
Oplismenopsis
Oplismenus
Oryzidium
Otachyrium
Ottochloa
Panicum
Paratheria
Parectenium
Paspalidium
Paspalum
Pennisetum
Perulifera
Plagiantha
Plagiosetum
Poecilostachys
Pseudechinolaena
Pseudochaetochloa
Pseudoraphis
Reimarochloa
Reynaudia
Rhynchelytrum
Sacciolepis
Scutachne
Setaria
Setariopsis
Snowdenia
Spheneria
Spinifex
Steinchisma
Stenotaphrum
Stereochlaena
Streptolophus
Streptostachys
Taeniorhachis
Tarigidia
Tatianyx
Thrasya
Thrasyopsis
Thuarea
Thyridachne
Trachys
Tricholaena
Triscenia
Uranthoecium
Urochloa
Whiteochloa
Xerochloa
Yakirra
Yvesia
Zygochloa

In embodiments of the invention, the plant to be subjected to the method(s) of the invention can be one found in nature, a cultivated nontransgenic plant, or a plant that has been modified through genetic means, such as, for example, a transgenic plant.

### Callus Source

Explant selections can be harvested from any portion of the plant that produces a callus or a mass of undifferentiated cells that can be cultured in vitro. For example, an explant selection can be obtained from the intercalary meristem tissue of a plant, immature inflorescences, or leaf meristematic tissue. In some embodiments, the explant selection can be obtained from a seed of a plant, or fragment or section thereof.

Prior to explant acquisition, the source tissue or seed can be subjected to a sterilization step to avoid microbial contamination in vitro. Sterilization can include rinsing in a bleach solution, such as, for example, a solution of from about 10% (v/v) to 100% (v/v), rinsing in an alcohol solution (e.g. ethanol), such as, for example, a solution of from about 50% (v/v) to 95% (v/v), and/or rinsing in sterile deionized water. The sterilization step can take place at any temperature that is not lethal to the plant material, preferably from about 20° C. to about 42° C.

Dry explants (explants that have been excised from seed under low moisture conditions) or dried wet explants (explants that have been excised from seed following hydration/imbibition and are subsequently dehydrated and stored) of various ages can be used. In some embodiments, explants are relatively “young” in that they have been removed from seeds for less than a day, for example, from about 1 to 24 hours, such as about 2, 3, 5, 7, 10, 12, 15, 20, or 23 hours prior to use. In some embodiments, explants can be stored for longer periods, including days, weeks, months or even years, depending upon storage conditions used to maintain explant viability. Those of skill in the art can understand that storage times can be optimized such efficient callus formation can be obtained.

In some embodiments, a dry seed or an explant can first be primed, for example, by imbibition of a liquid such as water or a sterilization liquid, redried, and later used for production of callus tissue.

The explant can be recovered from a hydrated seed, from dry storable seed, from a partial rehydration of dried hydrated explant, wherein “hydration” and “rehydration” is defined as a measurable change in internal seed moisture percentage, or from a seed that is “primed;” that is, a seed that has initiated germination but has been appropriately placed in stasis pending favorable conditions to complete the germination process. Those of skill in the art will be able to use various hydration methods and optimize length of incubation time prior to callus tissue induction. The resulting novel explant is storable and can germinate and/or be used to induce callus formation when appropriate conditions are provided. Thus the new dry, storable meristem explant can be referred to as an artificial seed.

The explant selection is cultured in an appropriate plant culture medium for promotion of callus formation. For example, the plant culture medium can be MS/B5 medium (Murashige and Skoog. 1962. Physiol Plant 15:473-497; Gamborg et al. 1968. Exp Cell Res 50:151-158, each of which is incorporated herein by reference in its entirety) supplemented with auxins and nutrients, including amino acids, carbohydrates and salts. A variety of tissue culture media are known that, when supplemented appropriately, support plant tissue growth and development, including formation of callus tissue from explant selections. These tissue culture medium can either be purchased as a commercial preparation or custom prepared and modified by those of skill in the art. Examples of such media include, but are not limited to those described by Murashige and Skoog (1962. Physiol Plant 15:473-497); Chu et al. (1975. Scientia Sinica 18:659-668); Linsmaier and Skoog (1965. Physiol Plant 18:100-127); Uchimiya and Murashige (1962. Plant Physiol 15:73); Gamborg et al. (1968. Exp Cell Res 50:151-158); Duncan et al. (1985. Planta 165:322-332); Lloyd and McCown (1981. Proc-Int Plant Propagator's Soc 30:421-427); Nitsch and Nitsch (1969. Science 163:85-87); and Schenk and Hildebrandt (1972. Can J Bot 50:199-204); each of the foregoing is incorporated herein by reference in its entirety. Likewise, those of skill in the art can make derivations of these media, supplemented accordingly. Those of skill in the art are aware that media and media supplements such as nutrients and growth regulators for use in transformation and regeneration are often optimized for the particular target crop or variety of interest. Tissue culture media can be supplemented with carbohydrates such as, but not limited to, glucose, sucrose, maltose, mannose, fructose, lactose, galactose, and/or dextrose, or ratios of carbohydrates. Reagents are commercially available and can be purchased from a number of suppliers (see, for example Sigma Chemical Co., St. Louis, Mo.; and PhytoTechnology Laboratories, Shawnee Mission, Kans.). In addition suitable auxins can include, but are not limited to, dicamba, 2,4-dichlorophenoxyacetic acid (“2,4-D”), and the like. Callus induction formulations can depend on the explant selection and can be selected and optimized according to protocols that are well-known to those of skill in the art.

### Evaluation of Callus Formation

The ability of each genotype to produce calli is evaluated before the first subculture occurs. The most prolific cell lines can be determined by observing the number of explants per genotype that produce callus. A relative numerical scale can be applied to each callus after approximately 30 days. For example, a numerical scale can consist of a rating of 1 to 5, depending on the amount of the callus produced by the explant. An exemplary rating of 5 can indicate that the explant produces a large amount of callus tissue, whereas a rating of 1 is assigned to the explants that have very low amounts of visible callus production. After rating, each callus is removed and subcultured. The calli produced by each explant can be identified as an individual cell line. Subculturing of each callus can be conducted every two or three weeks, for example.

### Evaluation of Dose Response to Herbicide

The appropriate herbicide concentration used in screening for resistant calli is assessed by placing callus tissue of each genotype to be tested on a series of induction medium plates with varying concentrations of herbicide. The range of herbicide concentrations tested in the dose-response assay is preferably 0 to 15 times the predicted lethal dosage, more preferably 2 to 10 times the predicted lethal dosage, and typically about 3 to 5 times the predicted lethal dosage. The herbicide concentration to be used in screening for resistant calli can be 30-50% greater than the minimum dosage at which there is no growth of the control callus, as determined by the dose-response assay.

### Selection of Herbicide-Resistant Cells

To select for herbicide-resistant cells, mature callus tissue can be placed on callus induction medium containing the appropriate herbicide concentration, as determined by the dose-response assay. Calli can be subcultured to fresh plates as necessary during the screening process. After resistant calli are identified, they can be subcultured onto induction medium for additional growth, sufficient to support regeneration.

Regeneration of Herbicide-Resistant Cells into Whole Plants

Calli are removed from plant culture medium and plated on an appropriate regeneration medium. A variety of tissue culture media are known that, when supplemented appropriately, support plant tissue growth, development and regeneration. These tissue culture media can either be purchased as a commercial preparation or custom prepared and modified by those of skill in the art. Examples of such media include, but are not limited to those listed hereinabove. As a nonlimiting example, Paspalum vaginatum can be regenerated by placing calli of each resistant line on medium consisting of MS/B5 basal medium supplemented with 1.24 mg L⁻¹ CuSO₄, and 1.125 mg/L⁻¹ BAP (6-benzylaminopurine). The regeneration medium can depend on the plant tissue source, and selection of the appropriate regeneration medium and protocol for regeneration are known to those of skill in the art.

Regeneration can occur on either solid or liquid media in receptacles such as, for example, petri dishes, flasks, tanks, or any other suitable chamber for that is used for culturing. The receptacle can optionally be sealed (e.g. with filter tape) so as to facilitate gas exchange for the regenerating plants. Growth chamber conditions can be at between about 20° C. or less, to 40° C. or more. In some embodiments, suitable temperatures for growth can range from about 22° C. to 37° C., about 25° C. to 35° C., or about 28° C. to 32° C. Dark:light exposure can range from about 1 hour dark:23 hours light to about 12 hours dark, or more:12 hours light, or less. In some embodiments, dark:light exposure can range from about 2 hours dark:22 hours light, to about 10 hours dark:14 hours light, from about 4 hours dark:20 hours light, to about 8 hours dark:16 hours light. Dark:light exposure can be followed by any where between about 1 hour to 10 hours of darkness, about 2 hours to 8 hours of darkness, or about 4 hours to 6 hours of darkness. In some embodiments, the dark period can be followed by additional cycles of dark:light exposure followed by dark exposure in any combination suitable for regeneration. The appropriate light intensity is selected according to well-known protocols in the art to facilitate growth. For example, to facilitate growth and regeneration of Paspalum vaginatumi, light intensity approximately equivalent to that provided by General Electric (GE) cool white bulbs at an intensity of 66-95 μm⁻²s⁻¹ can be provided to the growing plants.

### Progeny of Regenerated Plants

Regenerated plants can be reproduced asexually or asexually. For example, regenerated plants can be self-pollinated. In some embodiments, pollen can be obtained from regenerated plants and crossed to seed-grown plants of another plant having a second desired trait. In some embodiments, pollen can be obtained from a plant having a second desired trait and used to pollinate regenerated plants. The progeny of the regenerated plants can be, for example, a seed or a propagative cutting, in which the herbicide resistance of the regenerated plant is inherited from the parent. In addition, regenerated plants can be self-crossed or sib-crossed to develop a line of plants homozygous for the resistance allele. In some cases such homozygous plants can have a higher level of resistance than the originally selected, heterozygous, plants.

Vegetative propagation can be accomplished by using sod, plugs, sprigs, and stolons. When applied to turfgrass varieties, vegetative propagation of such grasses produces progeny that are typically clonal (genetically identical). Clonal vegetative varieties produce a turf that is very uniform in appearance.

Certain varieties are propagated solely by vegetative means; exemplary varieties having this feature include ornamentals, small fruits, and trees.

### Molecular Characterization of Herbicide Resistance

Mutations leading to herbicide resistance in plants can be characterized by extraction and subsequent PCR amplification of DNA from plant tissue. Plant DNA can be extracted via any number of DNA extraction methods, such as the CTAB method (Lassner, et al., 1989. Plant Mol. Biol. Rep. 7:116-128, which is incorporated herein by reference in its entirety), an SDS-potassium-acetate method (Dellaporta et al. 1983. Plant Molecular Biology Reporter 1:19-21, which is incorporated herein by reference in its entirety), direct amplification of leaf tissues (Berthomieu and Meyer 1991. Plant Molecular Biology 17: 555-557, which is incorporated herein by reference in its entirety), a boiling method (Ikeda et al. 2001. Plant Molecular Biology Reporter 19(1): 27-32, which is incorporated by reference herein in its entirety), an alkali treatment method (Xin et al. 2003. BioTechniques 34:820-826, which is incorporated by reference herein in its entirety), FTA® cards, or any other effective DNA extraction protocol for plants. Primers used to initiate PCR amplification of the regions of DNA conferring herbicide resistance can be designed to match conserved flanking sequences of the highest number of related species possible.

Identification of Mutations Associated with Resistance to ACCase Inhibitor Herbicides

Plants identified as being resistant to ACCase inhibitor herbicides by the methods disclosed herein can be evaluated for genetic mutations within the ACCase gene. For example, in some embodiments, the genetic mutations can lead to mutations in the ACCase protein at residues Gln 1756, Ile 1781, Trp 1999, Trp 2027, Ile 2041, Asp 2078, Cys 2088, and/or Gly 2096. In some embodiments, substitutions at those residues can include, but are not limited to leucine, alanine, valine, cysteine, aspartic acid, glycine, arginine, and glutamic acid. In some embodiments, the amino acid substitutions within the ACCase protein can be, for example, Gln 1756 to Glu, Ile 1781 to Leu, Ile 1781 to Ala, Ile 1781 to Val, Trp 1999 to Cys, Trp 2027 to Cys, Ile 2041 to Asp, Ile 2041 to Val, Asp 2078 to Gly, Asp 2078 to Val, Cys 2088 to Arg, and/or Gly 2096 to Ala, and the like. In some embodiments, the amino acid substitutions can be a combination of two or more mutations at positions such as those described above, involving changes such as those described above. Likewise, in some embodiments, other conservative substitutions can be made at these positions and/or at other positions known to those of skill in the art to be positions of contact or interaction between an ACCase and an ACCase inhibitor.

Mutations in the ACCase gene that lead to amino acid substitutions in the ACC protein include those listed in Table C.

TABLE C
Summary of Amino Acid Substitutions Associated with ACCase Inhibitor
Herbicide Resistance

| Amino Acid Residue - Position in | | |
|---|---|---|
| the CT Domain of the plastidic | | |
| ACCase protein | Substitution | Reference |
| Isoleucine - 1781 | Leucine | Délye et al. (2002a, 2002b, 2002c) |
| | | Christoffers et al. (2002) |
| | | White et al. (2005) |
| | | Liu et al. (2007) |
| | Alanine | |
| | Valine | Collavo et al. (2007) |
| Tryptophan - 1999 | Cysteine | Liu et al. (2007) |
| Tryptophan - 2027 | Cysteine | Délye et al. (2005) |
| | | Liu et al. (2007) |
| Isoleucine - 2041 | Aspartic Acid | Délye et al. (2003) |
| | | Liu et al. (2007) |
| | Valine | Délye et al. (2003) |
| Aspartic Acid - 2078 | Glycine | Délye et al. (2005) |
| | | Liu et al. (2007) |
| | Valine | Collavo et al. (2007) |
| Cysteine - 2088 | Arginine | Yu et al. (2007) |
| Glycine - 2096 | Alanine | Délye et al. (2005) |
| Glutamine - 1756 | Glutamic Acid | Zhang and Powles (2006) |

In addition, ACCase herbicide resistance can be conferred by any conservative substitutions at any of the referenced amino acid positions. A table of conservative substitutions is provided in Table D.

TABLE D
Conservative amino acid substitutions

| | |
|---|---|
| Group 1 | Ile, Leu, Val, Ala, Gly |
| Group 2 | Trp, Tyr, Phe |
| Group 3 | Asp, Glu, Asn, Gln |
| Group 4 | Cys, Ser, Thr, Met |
| Group 5 | Pro |
| Group 6 | His, Lys, Arg |

### Evaluation of Whole Plant Resistance to Herbicide

Whole plant herbicide resistance can be evaluated by comparing the effects of herbicide exposure on herbicide-resistant cell lines with herbicide-susceptible controls. Herbicide exposure can be accomplished by treating herbicide-resistant plants and herbicide-susceptible control plants with varying rates of herbicide, ranging from 0 to 20 times the known lethal dose for the species of interest.

### Herbicide Resistance

Embodiments of the invention relate to methods and compositions as disclosed herein to develop herbicide resistance in plants for commercial applications. In embodiments of the invention, the plants are selected and identified for being resistant to ACCase inhibitor herbicides.

Acetyl co-enzyme A carboxylase (ACCase) is known to exist in two forms: eukaryotic and prokaryotic. The prokaryotic form is made up of four subunits, while the eukaryotic form is a single polypeptide with distinct functional domains (Harwood, et al. 1988. Plant Molecular Biology 39:101-138, which is incorporated herein by reference in its entirety). Acetyl-coenzyme A is carboxylated by ACCase to form malonyl-coenzyme A in the first committed step of lipid biosynthesis. ACCase is compartmentalized in two forms in most plants (Sasaki, et al. 1995. Plant Physiology 108:445-449, which is incorporated herein by reference in its entirety). The chloroplast is known to be the primary site of lipid synthesis; however, ACCase can be present in the cytosol as well. Most plants have the prokaryotic form in the chloroplast and the eukaryotic form in the cytosol. The tetrameric prokaryotic protein is coded for by four distinct genes, one being located in the chloroplast genome. The eukaryotic form is encoded by a nuclear gene approximately 12,000 bp in size (Podkowinski, et al. 1996. PNAS 93:1870-1874, which is incorporated herein by reference in its entirety). Grasses are unique in that eukaryotic forms of ACCase are found in both the cytosol and chloroplast (Sasaki, et al. 1995. supra). The plastidic and cytosolic eukaryotic forms of ACCase in grasses are very similar, as are the genes that code for them (Gornicki, et al. 1994. PNAS 91:6860-6864, which is incorporated herein by reference in its entirety). However, despite the fact that there is homology between the plastidic and cystolic eukaryotic forms of ACCase, the cystolic form is not affected by ACCase-inhibiting herbicides (Delye. 2005. Plant Physiology 137:794-806, which is incorporated herein by reference in its entirety).

Herbicides that act as acetyl-coenzyme A carboxylase (ACCase) inhibitors interrupt lipid biosynthesis in plants, which can lead to membrane destruction actively growing areas such as meristematic tissue. ACCase inhibitors are exemplified by the aryloxyphenoxypropionate (APP) chemical family, also known as FOPS, and the cyclohexandione (CHD) family, also known as DIMs.

Accordingly, embodiments of the invention are directed to plants selected for resistance to ACCase inhibitor herbicides and methods of identifying the same. In some embodiments, the plant is resistant to a cyclohexanedione herbicide, an aryloxyphenoxy proprionate herbicide, a phenylpyrazoline herbicide, or mixtures thereof. In some embodiments, the plant is resistant to at least one herbicide selected from the list provided in Table E.

TABLE E
Acetyl Coenzyme A Carboxlyase Inhibitors

| Herbicide Class | | | |
|---|---|---|---|
| (Synonyms) | Active Name | Synonyms | Example Products |
| Cyclohexanediones | Alloxydim | Carbodimedon, | Fervin, Kusagard |
| (CHDs, DIMs) | | Zizalon, | |
| | | BAS 90210H | |
| | Butroxydim | Butoxydim | Falcon |
| | Clethodim | Cletodime | Select; Prism; |
| | | | Centurion; Envoy |
| | Cloproxydim | | Selectone |
| | Cycloxydim | BAS 517H, BAS 517 | Focus; Laser; Stratos |
| | Profoxydim | Clefoxydim; | Aura |
| | | BAS 625 H | |
| | Sethoxydim | Cyethoxydim | Poast; Rezult; |
| | | | Vantage; |
| | | | Checkmate, Expand, |
| | | | Fervinal, Grasidim, |
| | | | Sertin |
| | Tepraloxydim | Caloxydim | Aramo; Equinox |
| | Tralkoxydim | Tralkoxydime; | Achieve; Splendor; |
| | | Tralkoxidym | Grasp |
| Aryloxyphenoxy | Chlorazifop | | |
| propionates (APPs, | Clodinafop | | Discover, Topik |
| FOPs) | Clofop | Fenofibric Acid | Alopex |
| | Cyhalofop | | Barnstorm; Clincher |
| | Diclofop | Dichlorfop; Illoxan | Hoelon; Hoegrass; |
| | | | Illoxan |
| | Fenoxaprop | Fenoxaprop-P | Option; Acclaim; |
| | | | Fusion w/ Fluazifop |
| | Fenthiaprop | Fenthioprop; | Taifun; Joker; Hoe |
| | | Fentiaprop | 35609 |
| | Fluazifop | Fluazifop-P | Fusilade DX; Fusion |
| | | | w/ Fenoxaprop |
| | Haloxyfop | Haloxyfop-P | Edge; Motsa; |
| | | | Verdict; Gallant |
| | Isoxapyrifop | | HOK-1566; RH- |
| | | | 0898 |
| | Metamifop | | |
| | Propaquizafop | | Correct; Shogun; |
| | | | Agil |
| | Quizalofop | Quizalofop-P; | Assure; Targa |
| | | Quizafop | |
| | Trifop | | |
| Phenylpyrazoline | Pinoxaden | Only known ACCase | Axial |
| (DENs) | | inhibitor in its class | |

Herbicidal cyclohexanediones include, but are not limited to, sethoxydim (2-[1-(ethoxyimino)-butyl]-5-[2-(ethylthio)propyl]-3-hydroxy-2-cylohexen-1-one, commerically available from BASF (Parsippany, N.J.) under the designation POAST™), clethodim ((E,E)-(±)-2-[1-[[(3-chloro-2-propenyl)oxy]imino]propyl]-5-[2-(ethylthio)propyl]-3-hydroxy-2-cyclohexen-1-one; available as SELECT™ from Chevron Chemical (Valent) (Fresno, Calif.)), cloproxydim ((E,E)-2-[1-[[(3-chloro-2-propenyl)oxy]imino]butyl]-5-[2-(ethylthio)propyl]-3-hydroxy-2-cyclohexen-1-one; available as SELECTONE™ from Chevron Chemical (Valent) (Fresno, Calif.)), and tralkoxydim (2-[1-(ethoxyimino)propyl]-3-hydroxy-5-mesitylcyclohex-2-enone, available as GRASP™ from Dow Chemical USA (Midland, Mich.)). Additional herbicidal cyclohexanediones include, but are not limited to, clefoxydim, cycloxydim, and tepraloxydim.

Herbicidal aryloxyphenoxy proprionates and/or aryloxyphenoxypropanoic acids exhibit general and selective herbicidal activity against plants. In these compounds, the aryloxy group can be phenoxy, pyridinyloxy or quinoxalinyl. Herbicidal aryloxyphenoxy proprionates include, but are not limited to, haloxyfop ((2-[4-[[3-chloro-5-(trifluoromethyl)-2-pyridinyl]oxy]phenoxy]-propanoic acid), which is available as VERDICT™ from Dow Chemical U.S.A. (Midland, Mich.)), diclofop (((±)-2-[4-(2,4-dichlorophenoxy)-phenoxy]propanoic acid), available as HOELON™ from Hoechst-Roussel Agri-Vet Company (Somerville, N.J.)), fenoxaprop ((±)-2-[4-[(6-chloro-2-benzoxazolyl)oxy]phenoxy]propanoic acid; available as WHIP™ from Hoechst-Roussel Agri-Vet Company (Somerville, N.J.)); fluazifop ((±)-2-[4-[[5-(trifluoromethyl)-2-pyridinyl]oxy]phenoxy]propanoic acid; available as FUSILADE™ from ICI Americas (Wilmington, Del.)), fluazifop-P((R)-2-[4-[[5-(trifluoromethyl)-2-pyridinyl]oxy]phenoxy]propanoic acid; available as FUSILADE 2000™ from ICI Americas (Wilmington, Del.)), quizalofop ((±)-2-[4-[(6-chloro-2-quinoxalinyl)-oxy]phenoxy]propanoic acid; available as ASSURE™ from E. I. DuPont de Nemours (Wilmington, Del.)), and clodinafop.

Analogs of Herbicidal Cyclohexanediones or Herbicidal Aryloxphenoxy Proprionates or Herbicidal Phenylpyrazolines

Included among the ACCase inhibitors are herbicides that are structurally related to the herbicidal cyclohexanediones, herbicidal aryloxyphenoxy proprionates, or herbicidal phenylpyrazolines, as herein disclosed, such as, for example, analogs, metabolites, intermediates, precursors, salts, and the like.

Transformation with a Gene of Interest

In the methods disclosed herein, particular fragments of DNA have been isolated and cloned into vectors for the purposes of transforming plant tissue or cells. For example, a 384 base pair fragment has been isolated from the ACCase gene of Line A (Examples), in which an isoleucine to leucine mutation at position 1781 of the ACCase protein (“Ile1781Leu” or “I1781L”) has been identified. Such identified fragments can be used for transformation of plant tissues and cells as disclosed herein.

Various methods have been developed for transferring genes into plant tissue, including, but not limited to, high velocity microprojection, microinjection, electroporation, direct DNA uptake and, bacterially-mediated transformation. Bacteria known to mediate plant cell transformation include a number of species of the Rhizobiaceae, including, but not limited to, Agrobacterium sp., Sinorhizobium sp., Mesorhizobium sp., and Bradyrhizobium sp. (e.g. Broothaerts et al., 2005. Nature 433:629-633 and U.S. Patent Application Publication 2007/0271627, each of which is incorporated herein by reference in its entirety). Targets for such transformation can be undifferentiated callus tissues, differentiated tissue, a population of cells derived from a specific cell line, and the like. Co-culture and subsequent steps can be performed in dark conditions, or in the light, e.g. lighted Percival incubators, for instance for 2 to 5 days (e.g. a photoperiod of 16 hours of light/8 hours of dark, with light intensity of ≧5 μE, such as about 5-200 μE or other lighting conditions that allow for normal plastid development) at a temperature of approximately 23° C. or less to 25° C., and can be performed at up to about 35° C. or 40° C. or more.

The vector containing the isolated DNA fragment can contain a number of genetic components to facilitate transformation of the plant cell or tissue and regulate expression of the structural nucleic acid sequence.

In some embodiments, the vector can contain a selectable, screenable, or scoreable marker gene. These genetic components are also referred to herein as functional genetic components, as they produce a product that serves a function in the identification of a transformed plant, or a product of agronomic utility. The DNA that serves as a selection or screening device can function in a regenerable plant tissue to produce a compound that would confer upon the plant tissue resistance to an otherwise toxic compound. A number of screenable or selectable marker genes are known in the art and can be used in the present invention. Genes of interest for use as a marker would include but are not limited to GUS, green fluorescent protein (GFP), luciferase (LUX), and the like. Additional exemplary markers are known and include β-glucuronidase (GUS) that encodes an enzyme for various chromogenic substrates (Jefferson et al. 1987. Biochem Soc Trans 15:7-19; Jefferson et al. 1987. EMBO J. 6:3901-3907, each of which are incorporated herein by reference in its entirety); an R-locus gene, that encodes a product that regulates the production of anthocyanin pigments (red color) in plant tissues (Dellaporta et al. 1988. In: Chromosome Structure and Function: Impact of New Concepts. 18^{th} Stadler Genetics Symposium 11:283-282, which is incorporated herein b reference in its entirety); a β-lactamase gene (Sutcliffe et al. 1978. Proc Natl Acad Sci USA 75:3737-3741, which is incorporated herein by reference in its entirety); a gene that encodes an enzyme for that various chromogenic substrates are known (e.g., PADAC, a chromogenic cephalosporin); a luciferase gene (Ow et al. 1986. Science 234:856-859, which is incorporated herein by reference in its entirety); a xy1E gene (Zukowsky et al. 1983. Proc Natl Acad Sci USA 80:1101-1105, which is incorporated herein by reference in its entirety) that encodes a catechol dioxygenase that can convert chromogenic catechols; an α-amylase gene (Ikatu et al. 1990. Bio/Technol 8:241-242, which is incorporated herein by reference in its entirety); a tyrosinase gene (Katz et al. 1983. J Gen Microbiol 129:2703-2714, which is incorporated herein by reference in its entirety) that encodes an enzyme capable of oxidizing tyrosine to DOPA and dopaquinone that in turn condenses to melanin; green fluorescence protein (Elliot et al. 1999. Plant Cell Rep 18:707-714, which is incorporated herein by reference in its entirety) and an α-galactosidase. As is well known in the art, other methods for plant transformation can be utilized, for instance as described by Miki et al. (1993. In: Methods in Plant Molecular Biology and Biotechnology, Glick and Thompson (eds.), CRC Press, Inc.: Boca Raton, pp. 67-88, which is incorporated herein by reference in its entirety), including use of microprojectile bombardment (e.g. U.S. Pat. No. 5,914,451; McCabe et al. 1991. Bio/Technology 11:596-598; U.S. Pat. No. 5,015,580; U.S. Pat. No. 5,550,318; and U.S. Pat. No. 5,538,880; each of the foregoing is incorporated herein by reference in its entirety).

Transgenic plants can be regenerated from a transformed plant cell by methods and compositions known in the art. For example, a transgenic plant formed using Agrobacterium transformation methods typically contains a single simple recombinant DNA sequence inserted into one chromosome and is referred to as a transgenic event. Such transgenic plants can be referred to as being heterozygous for the inserted exogenous sequence. A transgenic plant homozygous with respect to a transgene can be obtained by sexually mating (selfing) an independent segregant transgenic plant that contains a single exogenous gene sequence to itself, for example an R₀ plant, to produce R₁ seed. One fourth of the R₁ seed produced will be homozygous with respect to the transgene. Germinating R₁ seed results in plants that can be tested for zygosity, typically using a SNP assay or a thermal amplification assay that allows for the distinction between heterozygotes and homozygotes (i.e., a zygosity assay). Alternatively, R₂ progeny can be developed and tested from several R₁ plants, wherein a homogeneous R₂ progeny, with all individuals resistant, is indicative of a homozygous R₁ parent.

To confirm the presence of the exogenous DNA or “transgene(s)” in the transgenic plants, a variety of assays can be performed. Such assays include, for example, “molecular biological” assays, such as Southern and northern blotting and PCR, INVADER™ assays; “biochemical” assays, such as detecting the presence of a protein product, e.g., by immunological means (ELISAs and western blots) or by enzymatic function; plant part assays, such as leaf or root assays; and also, by analyzing the phenotype of the whole regenerated plant.

Once a mutation has been selected for and confirmed in a plant, or once a transgene has been introduced into a plant, that mutation or transgene can be introduced into any plant that is sexually compatible with the first plant by crossing, without the need for directly selecting mutants in, or transforming, the second plant. Therefore, as used herein the term “progeny” can denote the offspring of any generation descended from a parent plant prepared in accordance with the instant invention, wherein the progeny comprises a desired genotype or phenotype, whether transgenic or non-transgenic. A “transgenic plant,” depending upon conventional usage and/or regulatory definitions, can thus be of any generation. “Crossing” a plant to provide a plant line having one or more selected mutations, phenotypes, and/or added transgenes or alleles relative to a starting plant line can result in a particular sequence being introduced into a plant line by crossing a starting or base plant line with a donor plant line that comprises a mutant allele, a transgene, or the like. To achieve this one can, for example, perform the following steps: (a) plant seeds of the first (starting line) and second (donor plant line that comprises a desired transgene or allele) parent plants; (b) grow the seeds of the first and second parent plants into plants that bear flowers; (c) pollinate a flower from the first parent plant with pollen from the second parent plant; and (d) harvest seeds produced on the parent plant bearing the fertilized flower.

### Methods of Controlling Weedy Grasses and Selectively Growing Herbicide-Resistant Plants

Exclusion of undesirable weedy grasses can be accomplished by treating the area in which exclusive growth of resistant plant species is desired, with herbicides to which resistance has been established. Accordingly, embodiments of the invention also relate to methods of controlling weeds in the vicinity of an herbicide-resistant plant identified by the methods disclosed herein, including: contacting at least one herbicide to the weeds and to the herbicide-resistant plant, wherein the at least one herbicide is contacted to the weeds and to the plant at a rate sufficient to inhibit growth or cause death of a non-selected plant of the same species and/or of a weed species desired to be suppressed. The non-selected plant typically is non-resistant to the herbicide.

In some embodiments, the herbicide can be contacted directly to the herbicide-resistant plant and to the weeds. For example, the herbicide can be dusted directly over the herbicide-resistant plant and the weeds. Alternatively, the herbicide can be sprayed directly on the herbicide-resistant plant and the weeds. Other means by which the herbicide can be applied to the herbicide-resistant plant and weeds include, but are not limited to, dusting or spraying over an area or plot of land containing the herbicide-resistant plant and the weeds.

In some embodiments, the herbicide can be contacted or added to a growth medium in which the herbicide-resistant plant and the weeds are located. The growth medium can be, but is not limited to, soil, peat, dirt, mud, or sand. In other embodiments, the herbicide can be included in water with which the plants are irrigated.

Typically, amounts of herbicide sufficient to cause growth or death of a non-resistant or non-selected plant ranges from about 2 μM or less to about 100 μM or more of herbicide concentration. In some embodiments, a sufficient amount of herbicide ranges from about 5 μM to about 50 μM of herbicide concentration, from about 8 μM to about 30 μM of herbicide concentration, or from about 10 μM to about 25 μM of herbicide concentration. Alternatively, amounts of herbicide sufficient to cause growth or death of a non-resistant plant ranges from about 25 grams of active ingredient per hectare (g ai ha⁻¹) to about 6500 g ai ha⁻¹ of herbicide application. In some embodiments, a sufficient amount of herbicide ranges from about 50 g ai ha⁻¹ to about 5000 g ai ha⁻¹ of herbicide application, about 75 g ai ha⁻¹ to about 2500 g ai ha⁻¹ of herbicide application, about 100 g ai ha⁻¹ to about 1500 g ai ha⁻¹ of herbicide application, or about 250 g ai ha⁻¹ to about 1000 g ai ha⁻¹ of herbicide application.

### Marker-Assisted Selection Methods

Marker-assisted selection (MAS), also known as molecular breeding or marker-assisted breeding (MAB), refers to to the process of selecting a desired trait or desired traits in a plant or plants by detecting one or more markers in the plant, where the marker is in linkage with the desired trait. In some embodiments, the marker used for MAS is a molecular marker. In other embodiments, it is a phenotypic marker, as discussed above.

In molecular breeding programs, genetic marker alleles can be used to identify plants that contain a desired genotype at one marker locus, several loci, or a haplotype, and that would therefore be expected to transfer the desired genotype, along with an associated desired phenotype, to their progeny. Markers are useful in plant breeding because, once established, they are not subject to environmental or epistatic interactions. Furthermore, certain types of markers are suited for high throughput detection, enabling rapid identification in a cost effective manner.

Due to allelic differences in molecular markers, quantitative trait loci (QTL) can be identified by statistical evaluation of the genotypes and phenotypes of segregating populations. Processes to map QTL are well known in the art and described in, for example, WO 90/04651; U.S. Pat. No. 5,492,547, U.S. Pat. No. 5,981,832, U.S. Pat. No. 6,455,758; Flint-Garcia et al. 2003 Ann. Rev. Plant Biol. 54:357-374, each of the foregoing which is incorporated herein by reference in its entirety. Using markers to infer phenotype in these cases results in the economization of a breeding program by substitution of costly, time-intensive phenotyping with genotyping. Marker approaches allow selection to occur before the plant reaches maturity, thus saving time and leading to efficient use of plots. Selection can also occur at the seed level such that preferred seeds are planted (U.S. Patent Publication No. 2005/000213435 and U.S. Patent Publication No. 2007/000680611, each of the foregoing which is incorporated herein by reference in its entirety). Further, breeding programs can be designed to explicitly drive the frequency of specific, favorable phenotypes by targeting particular genotypes (U.S. Pat. No. 6,399,855, which is incorporated herein by reference in its entirety). Fidelity of these associations can be monitored continuously to ensure maintained predictive ability and, thus, informed breeding decisions (U.S. Patent Application 2005/0015827, which is incorporated herein by reference in its entirety).

Accordingly, embodiments of the invention are directed to methods of marker-assisted breeding, including identifying a feature of interest for breeding and selection, wherein the feature is in linkage with an ACCase gene, providing a first plant carrying an ACCase sequence variant capable of conferring upon the plant resistance to an ACCase-inhibitor herbicide, wherein the plant further comprises the feature of interest, breeding the first plant with a second plant, identifying progeny of the breeding step as having the ACCase sequence variant, and selecting progeny likely to have the feature of interest based upon the identifying step. The feature of interest can be any one or more selected from the group of: herbicide tolerance, disease resistance, insect of pest resistance, altered fatty acid, protein or carbohydrate metabolism, increased growth rates, enhanced stress tolerance, preferred maturity, enhanced organoleptic properties, altered morphological characteristics, sterility, other agronomic traits, traits for industrial uses, or traits for improved consumer appeal.

In some embodiments, nucleic acid-based analyses for the presence or absence of the genetic polymorphism can be used for the selection of seeds or plants in a breeding population. The analysis can be used to select for genes, QTL, alleles, or genomic regions (haplotypes) that comprise or are linked to a genetic marker. For example, the marker can be the ACCase sequence variant that includes a variation corresponding to at least one amino acid position in the ACCase protein selected from the group of: Gln 1756, Ile 1781, Trp 1999, Trp 2027, Ile 2041, Asp 2078, Cys 2088 and Gly 2096. In some embodiments, the variation can be at least one selected from the group of: Gln1756Glu, Ile1781Leu, Ile1781Ala, Ile1781Val, Trp1999Cys, Trp2027Cys, Ile2041Asp, Ile2041Val, Asp2078Gly, Asp2078Val, Cys2088Arg and Gly2096Ala. Nucleic acid analysis methods are known in the art and include, but are not limited to, PCR-based detection methods (for example, TaqMan assays), microarray methods, and nucleic acid sequencing methods. In some embodiments, the detection of polymorphic sites in a sample of DNA, RNA, or cDNA can be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis, fluorescence detection methods, or other means. Thus, amplification assays, the oligonucleotides used in such assays, and the corresponding nucleic acid products produced by such assays can also be used in a marker-assisted breeding program to select for progeny having the desired trait or traits by selective breeding.

Likewise, MAS based upon resistance to ACCase-inhibtor herbicides can be done on a purely phenotypic basis. Initially plants are bred and selected, or engineered, such that a trait of interest is in non-random associate (linkage) with an allele conferring ACCase-inhibitor-resistance. Then that plant can be crossed with a plant having other desirable trait(s). Plants displaying resistance to ACCase inhibitors will be presumed to also carry the trait that is linked to the resistance marker. The presumption will be stronger as the linkage is closer/higher. Thus, an ACCase-inhibitor-resistance allele can serve either as a phenotypic marker for MAS, by producing plants that, for example, survive an otherwise lethal dose of an ACCase inhibitor, or as a molecular marker due to the ease of detection of the sequence variant associated with the resistance allele. For example, herbicide resistance, which is associated with an ACCase sequence variance, can be assayed. The herbicide resistance trait can include resistance to any one or more herbicides selected from the group of: alloxydim, butroxydim, cloproxydim, profoxydim, sethoxydim, clefoxydim, clethodim, cycloxydim, tepraloxydim, tralkoxydim, chloraizfop, clodinafop, clofop, cyhalofop, diclofop, fenoxaprop, fenthiaprop, fluazafop-butyl, fluazifop, haloxyfop, isoxapyrifop, metamifop, propaquizafop, quizalofop, trifop and pinoxaden. Selection by application of an ACCase inhibtor herbicide and observance of resistance to the herbicide can be evaluated as herein described.

MAS protocols are well known in the art, and employ various markers as tools. For example, MAS is described in U.S. Pat. No. 5,437,697, U.S. Patent Publication No. 2005/000204780, U.S. Patent Publication No. 2005/000216545, U.S. Patent Publication No. 2005/000218305, U.S. Patent Publication no. 2006000504538, U.S. Pat. No. 6,100,030 and in Mackill (2008. Phil Trans R Soc B 363:557-572), each of the foregoing which is incorporated herein by reference in its entirety. Accordingly, a person of skill in the art can use the resistance phenotype or sequences of the invention as a tool in an MAS protocol to select for traits that are linked to an ACCase-inhibitor-resistance allele.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of embodiments of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Callus Production Obtained from Intercalary Meristem of a Plant

An exemplary explant selection is illustrated in FIG. 18. Explant tissue can be obtained from a shoot containing the uppermost three leaves. The shoot is cut below the lowest leaf node, and the top of each leaf can be trimmed to conserve space during the sterilization procedure. The sections are placed in a bleach solution (20% v/v), for approximately 10 minutes, followed by 10 minutes in 70% ethanol before being rinsed with sterile water. The outer (older) two leaves are removed, leaving the newest leaf on the stem remaining. The new leaf is sterilized in 20% bleach for 1 minute, 70% ethanol for 1 minute, and subsequently rinsed in sterile water. The base of the leaf, next to the node, is the intercalary meristem. The lower 5 mm of this section is removed and plated on callus induction medium containing MS basal salts (Murashige and Skoog. 1962. Physiol Plant 15:473-497, which is incorporated herein by reference in its entirety) supplemented with B5 vitamins (Gamborg et al. 1968. Exp Cell Res 50:151-158, which is incorporated herein by reference in its entirety), 2,4-dichlorophenoxyacetic acid (“2,4-D”), sucrose, and adjusted to a pH of 8.5. The plated explants are placed in the dark at a temperature of 27° C.

TABLE 1
Callus induction medium

| Component | Concentration (per liter of medium) |
|---|---|
| MS basal salts | (Murashige and Skoog. 1962. supra) |
| B5 vitamins | (Gamborg et al. 1968. supra) |
| 2,4-D | 2 mg |
| Sucrose | 30 g |
| Gelzan ® | 2 g |

### Example 2

### Callus Production Obtained from Immature Inflorescences of Paspalum

Immature inflorescences were harvested from greenhouse grown plants prior to emergence and used as a source of explant tissue for generation of callus. The two spikes were separated and surface sterilized with 10% (v/v) bleach with several drops of Tween 80 for 10 minutes and rinsed with sterile water prior to plating on MS medium with B5 vitamins (Murashige and Skoog. 1962. supra; Gamborg et al. 1968. supra) and 2 mg/L 2-4,D. Explant tissue from 10 genotypes was obtained, including eight experimental lines from the University of Georgia Seashore Paspalum Breeding Program, one collected ecotype (Mauna Kea (PI 647892)), and the commercial seeded variety ‘Seaspray’. Four explants were placed on each plate, and the plates were sealed with Nescofilm™ (Karlan Research Products Co; Cottonwood, Ariz.). The explants were placed in the dark at 27° C. A total of 21 cell lines were generated from these 10 genotypes between (Table 2). Each generated callus was given a cell line designation based on the genotype and the date the explant tissue was placed on induction medium.

TABLE 2
Summary of in vitro callus generation and selection for mutations
conferring sethoxydim resistance in seashore paspalum

| | | | Calli | | SR | Positive |
|---|---|---|---|---|---|---|
| Cell | | Cell Line | Through | SR | Regen- | for 1781 |
| Line | Genotype | Initiation | Selection | Calli | erating | Mutation |
| 1 | Mauna Kea | 28 Nov 07 | 225 | 0 | 0 | 0 |
| 2 | Mauna Kea | 5 Dec 07 | 1350 | 3 | 0 | 0 |
| 3 | Mauna Kea | 12 Dec 07 | 225 | 0 | 0 | 0 |
| 4 | Mauna Kea | 9 Jan 08 | 1125 | 0 | 0 | 0 |
| 5 | Mauna Kea | 12 Jan 08 | 2025 | 29 | 2 | 2 |
| 6 | Mauna Kea | 21 Jan 08 | 450 | 7 | 0 | 0 |
| 7 | Mauna Kea | 6 Mar 08 | 1350 | 2 | 0 | 0 |
| 8 | Mauna Kea | 20 Mar 08 | 675 | 0 | 0 | 0 |
| 9 | Seaspray | 12 Jan 08 | 225 | 0 | 0 | 0 |
| 10 | 03-527.8 | 8 Jan 08 | 1575 | 0 | 0 | 0 |
| 11 | 03-527.8 | 21 Jan 08 | 900 | 0 | 0 | 0 |
| 12 | 03-527.8 | 16 May 08 | 225 | 0 | 0 | 0 |
| 13 | 03.539.13 | 6 Mar 08 | 3825 | 11 | 0 | 0 |
| 14 | 03.539.13 | 13 Mar 08 | 1800 | 7 | 0 | 0 |
| 15 | 05-025-164 | 20 Mar 08 | 675 | 0 | 0 | 0 |
| 16 | 05-025-164 | 9 Apr 08 | 450 | 2 | 0 | 0 |
| 17 | 05-025-181 | 4 Mar 08 | 450 | 1 | 0 | 1 |
| 18 | 03-107C-1 | 4 Mar 08 | 450 | 0 | 0 | 0 |
| 19 | 03-098E-3 | 4 Mar 08 | 900 | 2 | 1 | 0 |
| 20 | 03-134F.17 | 4 Mar 08 | 225 | 0 | 0 | 0 |
| 21 | 03.525.22 | 20 Apr 08 | 1125 | 1 | 0 | 0 |
| Total | | | 20250 | 65 | 3 | 3 |

### Example 3 Dose-Response Curve of Paspalum to Herbicide

The dose response of paspalum tissue in culture to sethoxydim rate was determined using callus tissue generated from the variety ‘Seaspray’ as a model cultivar. Effect of sethoxydim concentration on callus growth was determined by placing callus tissue from ‘Seaspray’ on MS/B5 medium (Murashige and Skoog. 1962. supra; Gamborg et al. 1968. supra) containing 2 mg/L 2-4,D and one of eight concentrations of sethoxydim. Herbicide rates were replicated 6 times and included concentrations of 0, 2.5, 5, 7.5, 10, 25, 50, and 100 μM sethoxydim. Sethoxydim was diluted in methanol and added after the autoclaved medium was cooled to approximately 55° C. (in order to prevent loss of activity from heat degradation). The medium was protected from photo-degradation by wrapping containers in aluminum foil prior to storage.

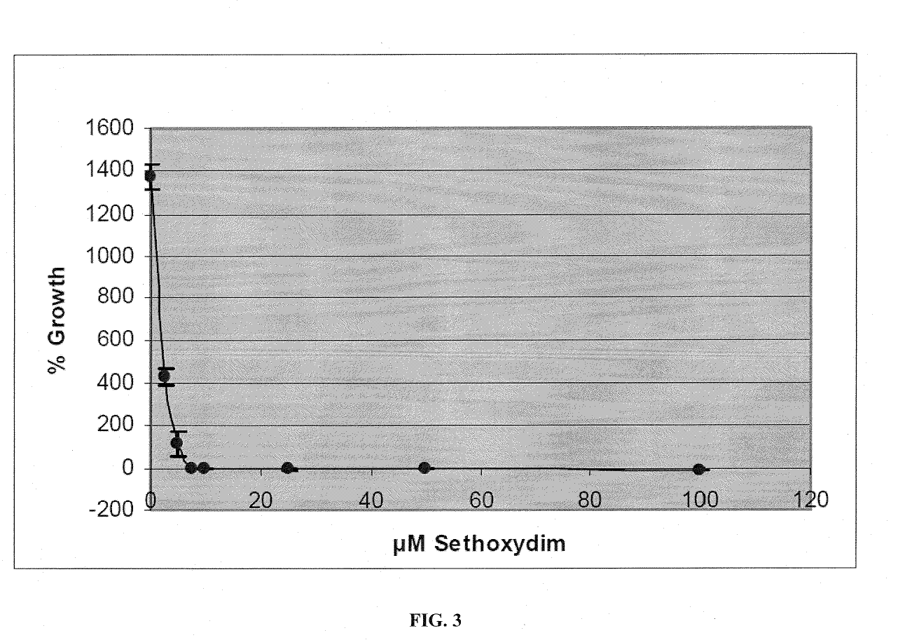

To measure callus growth, 0.5 gram of callus tissue was weighed, separated into nine equal pieces and placed in a 3×3 pattern on the solid medium of each plate. Six replicate plates for each of the eight sethoxydim concentrations was distributed on a rack in a growth room in a completely randomized design. At 21 days after plating, the tissue from each plate was weighed and recorded. For subculture, 0.5 gram from each plate was obtained for the next growth period. This process was continued for nine weeks, providing three growth measurements for each plate. The weight from each plate at each measurement point (3 weeks, 6 weeks, and 9 weeks) was divided by the initial weight to obtain the comparative increase in mass. Callus growth for each herbicide rate averaged over the three consecutive subcultures was used to discern an appropriate concentration for selection of mutants. Callus growth in response to sethoxydim concentration was fitted to a negative exponential decay function using non-linear regression (SAS Institute, Inc. 2008. SAS OnlineDoc® 9.2. Cary, N.C.). The lowest herbicide rate to totally inhibit callus growth was 7.5 μM sethoxydim. To ensure efficacy, a concentration of 10 μM sethoxydim was chosen for selection of resistant cells (FIG. 3).

### Example 4

### Selection of Sethoxydim-Resistant Cell Lines

Selection of sethoxydim resistant (SR) cells was performed by placing approximately six-month old callus tissue on callus induction medium (Example 1) containing 10 μM sethoxydim. Large plates (245×245 mm in size) were used to efficiently screen greater numbers of cells. Callus tissue approximately 4-mm in diameter was placed in a 15×15 grid, giving a total of 225 calli per plate. Calli were subcultured three times at three-week intervals (Example 3) for a total selection period of nine weeks. Resistant calli were subcultured into 100×15 mm petri dishes containing callus induction medium (Example 1) supplemented with 10 μM sethoxydim for one month in order to obtain sufficient callus. This provided a total selection time of 12 weeks or more.

A total of 20,250 calli were screened. The selection process resulted in 65 sethoxydim-resistant (SR) lines, representing a mutation rate of one resistance event per 312 calli. The six cell lines that produced SR calli were: Mauna Kea, GA 05-025-164, UGA03.539.13, UGA05.025.181, UGA03.525.22, and UGA03.09E-3. The frequency of SR calli was low in all genotypes and ranged from 0 to 0.0051. Even though the probability of recovering a SR line was low for all genotypes, the number of SR lines recovered varied and ranged from zero to as high as nine per plate of 225 calli. Statistical analysis for differences in the probability of obtaining a resistant calli event indicated no significant differences (p=0.35) among genotypes. Resistant calli were given SR designations, removed from selection medium, and subcultured to increase tissue prior to regeneration.

### Example 5

### Regeneration of Sethoxydim Resistant Lines

Regeneration was attempted on all resistant calli. The regeneration medium used was MS/B5 medium (Murashige and Skoog. 1962. supra; Gamborg et al. 1968. supra) supplemented with 1.24 mg/L CuSO₄, and 1.125 mg/L 6-benzylaminopurine (BAP) (Altpeter, et al. 2005. International Turfgrass Society Research Journal 10:485-489, which is incorporated herein by reference in its entirety). Calli of each sethoxydim resistant (SR) line were placed in a 4×4 grid on five plates, with each callus having an approximate diameter of 4 mm in size. The plates were then placed in a growth chamber at 25° C. with a 1-h dark:23-h light photoperiod, wherein the light intensity was provided at 66-95 μmol photons m⁻²s⁻¹ by cool white fluorescent tubes. All plates were evaluated for regeneration at the end of a 30-day period. If shoots appeared the cell lines were subcultured for an additional month on regeneration medium.

After shoot development, roots were induced by placing tissue on MSO medium (as listed in Table 3 below) without growth regulators. When root growth was adequate (about 30 days), plants were removed from the medium and placed directly in pots containing a 1:1 mix of Fafard® 3B (Agawam, MS) mix and sand. The potted plants were then transferred to a greenhouse with 10 hour light, 14 hour dark photoperiods at 24° C. to 32° C.

TABLE 3
MSO medium for root induction

| Component | Concentration (per liter of medium) |
|---|---|
| MS basal salts | (Murashige and Skoog. 1962. supra) |
| B5 vitamins | (Gamborg et al. 1968. supra) |
| Sucrose | 30 g |
| Gelrite ® | 2 g |

Two of the 65 SR cell lines were lost prior to regeneration, thus, of the 63 SR lines remaining, three lines were regenerated: Line A, Line B, and Line C. Lines A and B originated from the same cell line derived from Mauna Kea initiated on 12 Jan. 2008, while Line C originated from experimental line UGA 03-098E-3 initiated on 4 Mar. 2008. The callus tissue of the three lines that regenerated was dense and yellow compared to a majority of the lines, which were white and soft.

### Example 6

### Molecular Characterization of Sethoxydim Resistant Lines

Once SR paspalum lines were selected, the mutation causing the resistance was characterized. DNA was extracted from the callus or leaf tissue of regenerated plants using the CTAB method (Lassner et al. 1989. Plant Mol Biol Report 7:116-128, which is incorporated herein by reference in its entirety). Acetyl coenzyme A carboxylase (ACCase) amino acid sequences (Délye, et al. 2005. Weed Research 45:323-330, which is incorporated herein by reference in its entirety) were used to determine homologous regions among species. The nucleotide sequence from Setaria viridis ACCase (GenBank AF294805) (Délye, et al. 2002. Planta 214:421-427, which is incorporated herein by reference in its entirety) was used to design primers that amplify the homologous region in seashore paspalum, and individual bases were changed to match the highest number of grass species possible as determined by the BLAST function of GenBank. The resulting primers amplify a 384 base pair fragment of the ACCase gene that spans the A to T transversion which causes the Ile to Leu substitution at the 1781 position. The primers were designated SV384F (5′ CGGGGTTCAGTACATTTAT 3′, SEQ ID NO: 1) and SV348R (5′ GATCTTAGGACCACCCAACTG 3′, SEQ ID NO: 2). The annealing temperature was 53° C. with an extension time of 30 seconds and 35 cycles. The primers developed for sequencing the 2078 position of the ACCase gene were designated SVAC2F (5′ AATTCCTGTTGGTGTCATAGCTGTGGAG 3′, SEQ ID NO: 3) and SVAC1R (5′ TTCAGATTTATCAACTCTGGGTCAAGCC 3′, SEQ ID NO: 4), and the PCR conditions used to amplify this segment were the same as the conditions to used to amplify 1781. The SVAC primers amplify a 520-bp fragment that spans the coding region of position 2078 in the ACCase gene.

### Example 7

### Identification of Sethoxydim Resistant Cell Lines and Regeneration of Sethoxydim Resistant Paspalum from Cell Lines

Table 2 summarizes the selection process to date. To date, 65 sethoxydim resistant cell lines have been produced. The frequency of resistant calli formation was 1 per 312 calli undergoing the full selection process. The frequency of regenerable sethoxydim resistant (SR) calli was 1 per 32.5 resistant calli. The frequency of SR lines that regenerated was 1 per 10,125 calli put through the selection process.

The average volume of a single callus cell was measured to be 1.3582×10⁻⁵ μL. This provides an approximation of 258,000 cells per 4 mm-diameter callus piece. Thus, the 20,250 calli put through selection contained approximately 5.2 billion cells. Assuming that only a single mutant cell was responsible for each SR cell line, the frequency of resistant cells in this experiment was one per 8×10⁷ cells. The frequency of obtaining the A to T mutation at the 1781 aa position was one in 1.74×10⁹.

To date, four SR calli, Line A, Line B, Line C and Line D have produced green plantlets, and two SR calli (Line A and Line B) have been established as viable plants. Lines A, B and D originated from the same cell line, Mauna Kea 12Jan. 2008, while Line C originated from experimental line UGA 03-098E-3 initiated on 4 Mar. 2008. Line A has been the most prolific in terms of regenerated plants, producing more than 500 individual plants. Line B has produced approximately 20 plants.

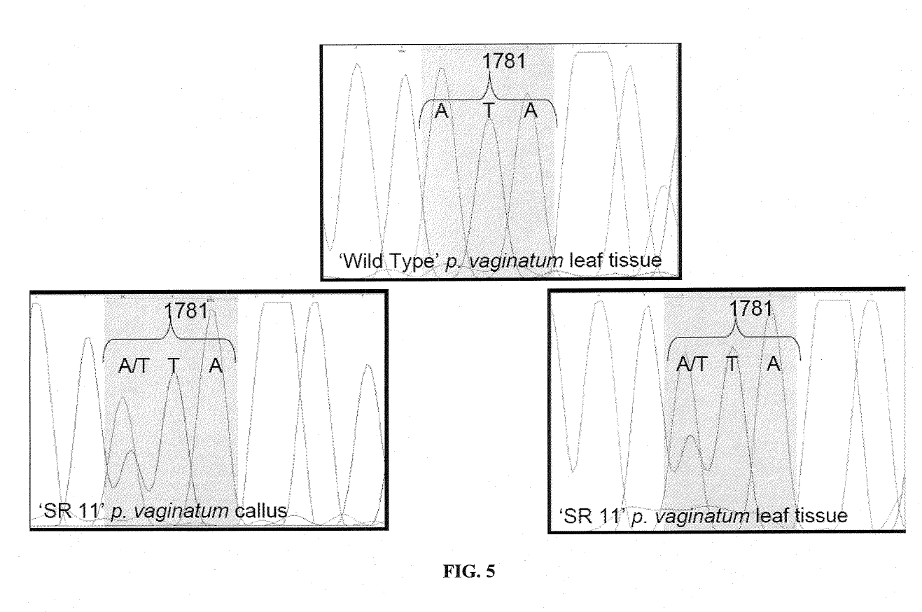

ACCase amplicons were obtained from 63 of the 65 SR lines, and only three lines, including Line A (FIG. 5), exhibited the A to T transversion at position 1781. The possibility exists that mutations at positions other than 1781 or 2078 also occurred in these SR cell lines. Resistant lines are heterozygous for the mutation, so the sequence chromatograms illustrate a double peak at the point of mutation, with one peak representing the wild-type allele, and the other the mutated allele. Of the two lines that produced viable plants, only Line A possesses the expected Ile to Leu mutation. The genetic sequence of the amplicon obtained for Line A is given below as SEQ ID NO: 5, with the highlighted and underlined codon indicating the Ile to Leu mutation. Line B has the wild-type sequence at position 1781. Since sethoxydim resistance can also be conferred by an Asp to Gly mutation at the 2078 position; DNA from Line B was analyzed for presence of this mutation, but neither line possessed it. The nature of sethoxydim resistance remains undetermined for Line B.

More that 500 Line A plants have been transplanted to soil. The regenerated plants of Line A were vegetatively increased for undergoing herbicide testing in order to confirm expression of sethoxydim resistance at the whole plant level.

SEQ ID NO: 5

GGCGATTGGGCCGAAGTCGCATGCTCCCGGCCGCCATGGCGGCCGCGGGA
ATTCGATACCCCTTTTTCAGTACATTTATCTGACTGAAGAAGATTATGCT
CGTATTAGCTCTTCTGTTATAGCACATAAGCTACAGCTGGACAGCGGTGA
AATTAGGTGGATTATTGACTCTGTTGTGGGCAAGGAGGATGGGCTTGGTG
TTGAGAATTTACATGGAAGTGCTGCTATTGCCAGTGCTTATTCTAGGGCA
TACGAGGAGACATTTACACTTACGTTCGTGACTGGGCGGACTGTAGGAAT
AGGAGCTTATCTTGCACGACTTGGTATACGGTGCATACAGCGTCTTGACC
AGCCCATTATTTTAACAGGGTTTTCTGCCCTGAACAAGCTTCTTGGGCGT
GAAGTTTACAGCTCCCACATGCAGTTGGGTGGTCCTAAGATCATGGCGAC
GAATGGTGTTGTCCACCTCACTGTTTCAGATGATCTTGAAGGTGTATCCA
GTATATTGAGGTGGCTCAGCTATGTTCCTGCCAACATTGGTGGACCTCTT
CCTATTACAAAACCTTTGGACCCACCGGACAGACCTGTTGCGTACATCCC
TGAGAACACATGCGATCCACGTGCAGCCATCCGTGGTGTAGATGACAGCC
AAGGGCAATGGTTGGGTGGTATGTTTGACAAAGACAGCTTTGTGGAGACA
TTTGAAGGATGGGCGAAAACAGTTGTCACTGGCAGGGCATAGCTTGGAGG
AATTCCTGTGGGTGTCATAGCTGTGGAGACACAGAACATGATGCAGCTCA
TCCCTGCTGATCCAGGCCAGCTTGATTCTCATGAGCGATCTGTTCCTCGG
GCTGAACAAGTGTGGTTCCCAGATTCTGCAACCAAGACTGCTCAAGCATT
GTTGGACTTCAACCGTGAAGGATTGCCTCTGTTCATCCTTGCTAACTGGA
GAGGTTTCTCTGGTGGACAAAGAGATCTCTTTGAAGGAATTCTTCAGGCT
GGGTCAACAATTGTTGAGAACCTTAGGACGTACAATCAACCTGCGTTTGT
CTACATTCCTATGGCTGGAGAGCTGCGTGGAGGAGCTTGGGTTGTGGTTG
ATAGCAAAATAA

A vector containing SEQ ID NO: 5 was deposited with the American Type Culture Collection, 10801 University Boulevard, Manassas, Va. 20110-2209 U.S.A. on Jun. ______, 2009 and assigned Accession No. ______.

### Example 8

### Evaluation of Whole Plant Resistance to Sethoxydim (Segment™ Herbicide)

Sethoxydim-resistant plants regenerated from a sethoxydim-resistant cell line, Line A, were tested for resistance at the whole plant level in a dose-response experiment conducted in a greenhouse. In this experiment, Line A was compared to two herbicide-susceptible controls; the original parent line, Mauna Kea (PT); and a Mauna Kea line regenerated from tissue culture (TTC). Plants were transplanted to Cone-tainers™ measuring 4×14 cm and tapering to 1 cm (Stuewe and Sons Inc., Corvallis, Oreg.) containing a 1:1 mix of Fafard® 3B mix and sand and placed on benches under sodium lights in a greenhouse with a 16 hour photoperiod maintained at 27/32° C. day/night for two weeks prior to treatment applications. Each of the three genotypes, Line A, PT and TC were treated with 0, 50, 100, 200, 400, 800, 1600, and 3200 g ai ha⁻¹ rates of sethoxydim using Segment™ herbicide (BASF Corp., Florham Park, N.J.). All herbicide rates were applied at a spray volume of 1871 ha⁻¹ in an experimental spray chamber, and after drying, returned to the greenhouse bench and maintained under the conditions described above. Visual estimates of crop injury were recorded at 7, 14, 21, and 28 days after treatment (DAT) using a scale of 0 to 100, where 0 equals no injury and 100 equals complete death. At 42 days after treatment, the above ground portion of all plants was harvested, dried for 48 hours at a temperature of 50° C., and weighed to determine plant dry weight. Treatments were arranged in a randomized complete block design. Only two replications of TCC were possible due to limited plant materials; otherwise four replications were used for the other two genotypes (PT and Line A). Data were first analyzed using a two-way analysis of variance and subsequently analyzed within herbicide rate. Differences among genotype means at each herbicide rate were determined using Fisher's Least Significant Difference (LSD).

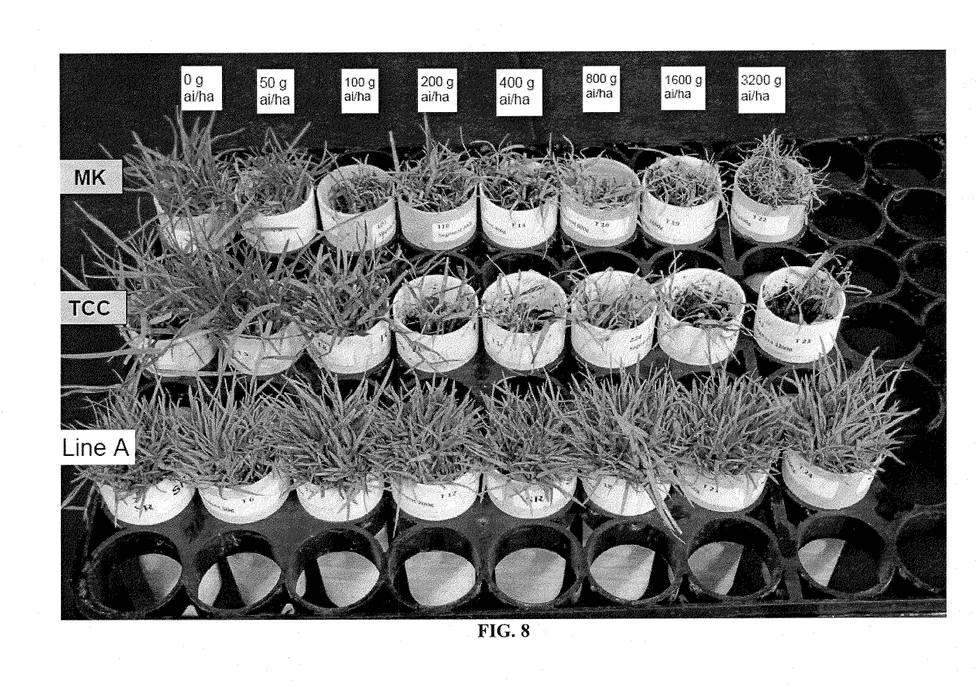

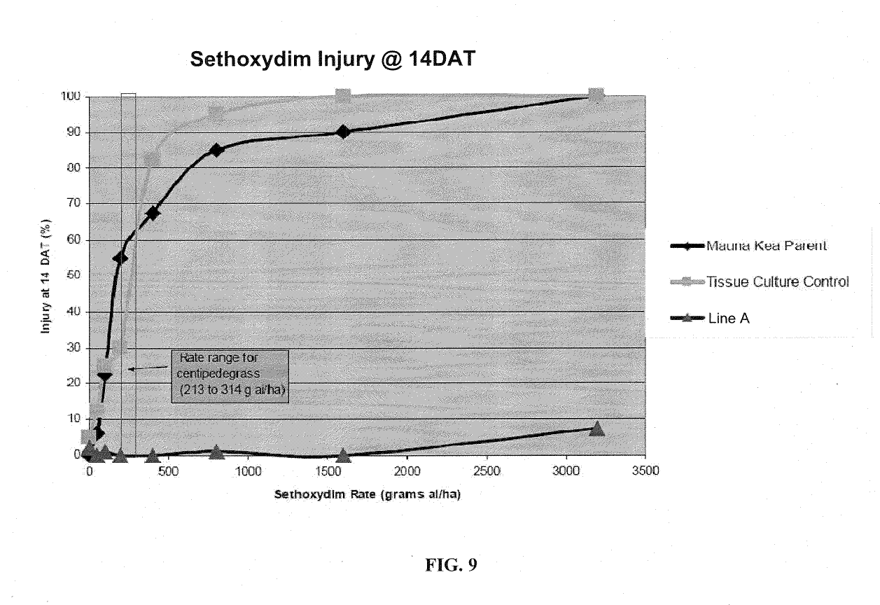

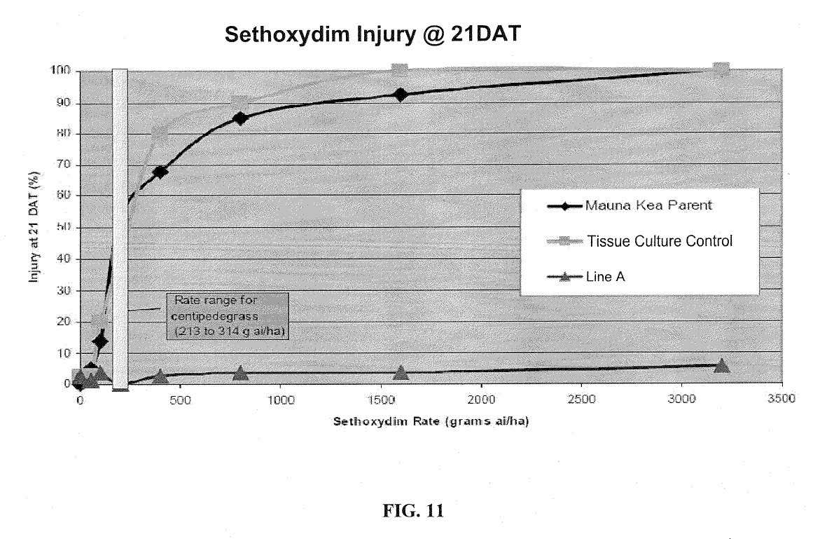

FIG. 9 illustrates the effect of sethoxydim rate on injury ratings of each of the three tested genotypes at 14 DAT. FIG. 11 illustrates the effect of sethoxydim rate on injury ratings of each of the three tested genotypes at 21 DAT. The two-way analysis of variance indicated significant genotype, herbicide rate, and genotype by herbicide rate effects for injury ratings at 7, 14, 21, and 28 days after treatment (data not shown). Line A showed excellent herbicide resistance, even at the highest rate of 3200 g ai ha⁻¹ (FIG. 8, Table 4). In contrast, both PT and TC had injury scores of 30 or greater at rates of 200 g ai ha⁻¹, and injury scores of 80% or greater at rates equal to or greater than 800 g ai ha⁻¹. When mean injury scores were compared for each of the three genotypes at each herbicide rate, Line A had significantly less injury than PT or TC at all rates above 100 g ai ha⁻¹ at all rating dates. The maximum injury score observed on Line A was 7.5% at 3200 g ai ha⁻¹, or 15 times greater dosage than the lowest labeled rate for centipedegrass, Eremochloa ophiuroides (Munro) Hack, a turfgrass species naturally tolerant to sethoxydim.

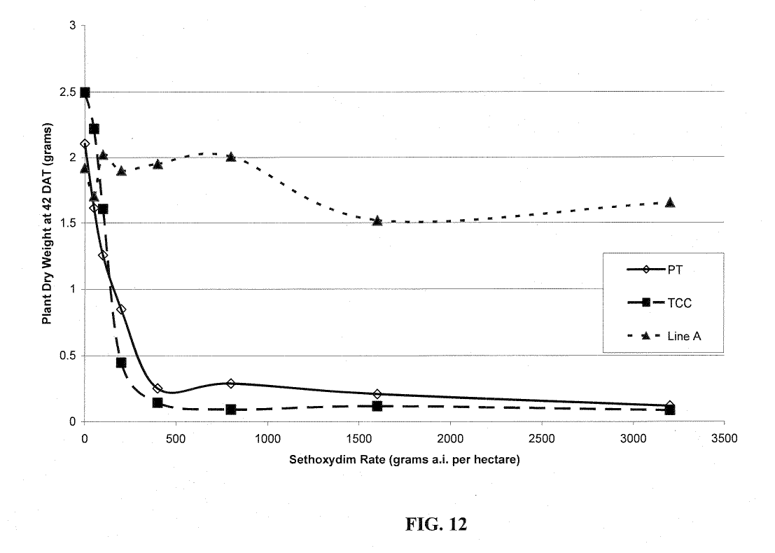

Mean dry weight of the three genotypes taken 42 DAT are presented in FIG. 12. Dry weights of the two susceptible lines, PT and TCC decreased in response to increasing sethoxydim rate while the dry weight of CLA remained relatively unchanged even at rates above 1600 g ai ha⁻¹.

Estimates of LD₅₀ for the three genotypes were 189, 276, and >3200 g ai ha⁻¹ for PT, TC, and Line A, respectively. These data provide evidence that the level of herbicide resistance present in Line A is more than adequate to provide effective control of susceptible weedy grasses without concerns over herbicide injury.

TABLE 4
Response of three genotypes of seashore paspalum to sethoxydim rate.

| | Plant Injury | | | | | | | | | | | | Dry Weight | | |
|---|---|---|---|---|---|---|---|---|---|---|---|---|---|---|---|
| Herbicide | 7 DAT² | | | 14 DAT | | | 21 DAT | | | 28 DAT | | | 42 DAT | | |
| Rate¹ | PT | TC | Line A | PT | TC | Line A | PT | TC | Line A | PT | TC | Line A | PT | TC | Line A |
| grams | % | | | | | | | | | | | | grams | | |
| 0 | 0.0a³ | 0.0a | 1.7a | 0.0a | 5.0a | 2.5a | 0.0a | 2.5a | 2.5a | 0.0a | 0.0a | 0.0a | 2.1a | 2.5a | 1.9a |
| 50 | 5.2a | 4.5a | 2.9a | 6.2b | 12.5ab | 0.0b | 5.0a | 2.5a | 1.2a | 1.2a | 0.0a | 0.0a | 1.6a | 2.2a | 1.7a |
| 100 | 7.9b | 18.3b | 0.8a | 22.5b | 25.0b | 1.2a | 13.8a | 20.0a | 3.8a | 6.2a | 7.5a | 2.5a | 1.3b | 1.6ab | 2.0a |
| 200 | 20.8b | 16.7b | 1.7a | 55.0b | 30.0b | 0.0a | 52.5b | 40.0b | 0.0a | 43.8b | 32.5b | 0.0a | 0.8b | 0.4b | 1.9a |
| 400 | 30.8b | 30.8b | 3.8a | 67.5b | 82.5b | 0.0a | 67.5b | 80.0b | 2.5a | 72.5b | 82.5b | 0.0a | 0.2b | 0.1b | 2.0a |
| 800 | 35.0b | 60.0c | 1.2a | 85.0b | 87.5b | 1.2a | 85.0b | 95.0b | 3.8a | 88.8b | 100.0c | 1.2a | 0.3b | 0.1b | 2.0a |
| 1600 | 40.8b | 43.3b | 4.3a | 90.0b | 100.0c | 0.0a | 92.5b | 100.0c | 3.8a | 92.5b | 100.0b | 1.2a | 0.2b | 0.1b | 1.5a |
| 3200 | 37.9b | 46.7b | 8.3a | 100.0b | 100.0b | 7.5a | 100.0b | 100.0b | 5.5a | 100.0b | 100.0b | 4.2b | 0.1b | 0.1b | 1.6a |

¹Grams a.i. ha⁻¹
²DAT = days after treatment.
³Means on the same row (herbicide rate) and within a measured variable group (i.e. 7 DAT) followed by the same letter are not considered to be significantly different at 0.05 according to a protected LSD.

### Example 9

### Evaluation of Whole Plant Resistance to Sethoxydim (Poast™ Herbicide)

A second greenhouse experiment was initiated to evaluate SR plants regenerated from a second sethoxydim-resistant cell line, Line B, for sethoxydim resistance at the whole plant level. In the previous experiment (Example 8), minor injury occurred on Line A at higher concentrations of Segment™ sethoxydim herbicide. These injury symptoms were more indicative of surfactant injury rather than sethoxydim injury. Accordingly, Poast™ herbicide, a formulation of sethoxydim that does not contain surfactant, was chosen to characterize the resistance level of Line B and to compare the level of sethoxydim resistance of Line B to Line A. In this experiment both Line A and Line B were compared to two herbicide-susceptible controls: the original parental line, Mauna Kea (PT); and a Mauna Kea line regenerated from tissue culture (TTC). Plants were transplanted to Cone-tainers™ measuring 4×14 cm and tapering to 1 cm (Stuewe and Sons Inc., Corvallis, Oreg.) containing a 1:1 mix of Fafard® 3B mix and sand and placed on benches under sodium lights in a greenhouse with a 16-h photoperiod maintained at 27/32° C. day/night for approximately two weeks prior to application of herbicide treatments.

Each of the four genotypes (Line A, Line B, PT and TCC) were treated with 0, 50, 100, 200, 400, 800, 1600, 3200 and 6400 g ai ha⁻¹ rates of sethoxydim using Poast™ herbicide (BASF Corp., Florham Park, N.J.). All herbicide rates were applied at a spray volume of 1871 ha⁻¹ in an experimental spray chamber, and after drying, the plants were returned to the greenhouse bench and maintained under the conditions described above. Visual estimates of crop injury were recorded at 16, 21, and 28 d after treatment (DAT) using a scale of 0 to 100, where 0 equals no injury and 100 equals complete death. The experiment was a four by nine factorial with four genotypes and nine herbicide rates. Treatments were arranged in a randomized complete block design. Four replications were used for all four genotypes. Data were first analyzed using a two-way analysis of variance (SAS, 2008) and subsequently analyzed within herbicide rate. Differences among genotype means at each herbicide rate were determined using Fisher's Least Significant Difference (LSD).

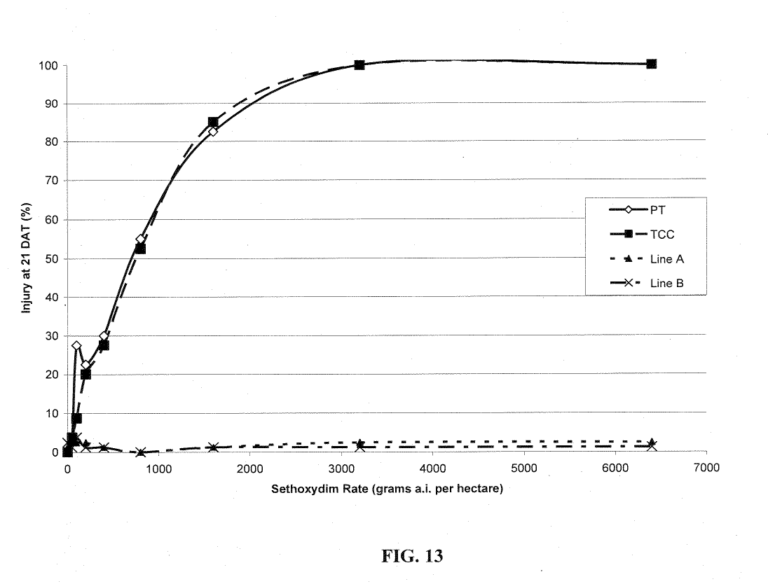

FIG. 13 illustrates the effect of sethoxydim rate on injury ratings of each of the four tested genotypes at 21 DAT. The two-way analysis of variance indicated significant genotype, herbicide rate, and genotype by herbicide rate effects for injury ratings at 16, 21, and 28 DAT (data not shown). Both Line A and Line B showed excellent herbicide resistance, even at the highest rate of 6400 g ai ha⁻¹ (FIG. 13). In contrast, both PT and TCC. had injury scores of 27 or greater at rates of 400 g ai ha⁻¹, and injury scores of 80% or greater at rates of 1600 g ai ha⁻¹ or more. When mean injury scores were compared for each of the four genotypes at each herbicide rate, Line A and Line B had significantly less injury than PT or TCC at all rates of above 200 g ai ha⁻¹ at all rating dates. The maximum injury score observed on Line A and Line B was less than 20% for all rates up to 6400 g ai ha⁻¹.

Estimates of LD₅₀ for the four genotypes were 720, 782, >6400, >6400 g ai ha⁻¹ for PT, TC, Line A, and Line B, respectively. These data provide strong evidence that the level of herbicide resistance present in both Line A and Line B is more than adequate to provide effective control of susceptible weedy grasses without concerns over herbicide injury.

### Example 10

### Cross-Resistance of Sethoxydim-Resistant Paspalum to Other ACCase Inhibitor Herbicides

Sethoxydim is a member of the class known as ACCase inhibiting herbicides. This family of herbicides is often divided into two groups, the cyclohexanediones (CHD), characterized by a cyclohexane ring, and commonly referred to as the “Dims”, and the aryloxyphenoxypropionate (APP) herbicides, commonly referred to as the “Fops”. Depending on structural and/or side chain similarities, resistance to sethoxydim can be indicative of resistance to a broad class of herbicides in the ACCase inhibitor family. For example, cross resistance to both CHD and APP herbicides has been reported in several weedy species of plants possessing the 1781 ILE to LEU mutation most commonly associated with sethoxydim resistance (Délye, 2005. Weed Science 53:728-746, which is incorporated herein by reference in its entirety). Accordingly, resistance of sethoxydim-resistant Lines A and B to other ACCase inhibiting herbicides was determined in a series of greenhouse experiments

In the experiments, both Line A and Line B were compared to two herbicide-susceptible controls; the original parental line, Mauna Kea (PT); and a Mauna Kea line regenerated from tissue culture (TTC). Plants were transplanted to Cone-tainers™ measuring 4×14 cm and tapering to 1 cm (Stuewe and Sons Inc., Corvallis, Oreg.) containing a 1:1 mix of Fafard® 3B mix and sand and placed on benches under sodium lights in a greenhouse with a 16-h photoperiod maintained at 27/32° C. day/night for approximately two weeks prior to application of herbicide treatments.

Each of the four genotypes, Line A, Line B, PT, TCC, were compared in three separate herbicide dose-response experiments. Herbicides tested included fluazifop-p-butyl (Fusilade II™) and fenoxaprop-p-ethyl (Acclaim Extra™). In the each of the experiments four replicates of each of the four genotypes was treated with nine rates of the appropriate herbicide. The fluazifop rates 0, 25, 50, 100, 200, 400, 800, 1600 and 3200 g ai ha⁻¹ rates of fluazifop-p-butyl using Fusilade II™ herbicide (Syngenta Crop Protection, Inc., Greensboro, N.C.). The fenoxaprop rates were 0, 25, 50, 100, 200, 400, 800, 1600 and 3200 g ai ha⁻¹ rates of fenoxaprop-p-ethyl using Acclaim Extra™ herbicide (Bayer Environmental Science, Montvale, N.J.). All herbicide rates were applied at a spray volume of 187 L ha⁻¹ in an experimental spray chamber, and after drying, the plants were returned to the greenhouse bench and maintained under the conditions described above. Visual estimates of crop injury were recorded at 21 and 28 days after treatment (DAT) using a scale of 0 to 100, where 0 equals no injury and 100 equals complete death. The experiment was a four by nine factorial with four genotypes and nine herbicide rates. Treatments were arranged in a randomized complete block design. Four replications were used for all four genotypes. Data were first analyzed using a two-way analysis of variance (SAS, 2008) and subsequently analyzed within herbicide rate. Differences among genotype means at each herbicide rate were determined using Fisher's Least Significant Difference (LSD).

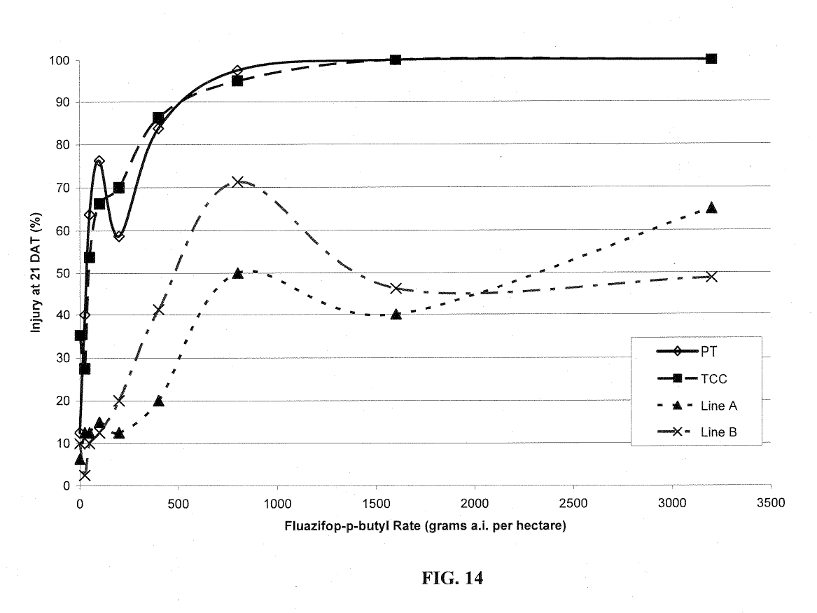

FIG. 14 illustrates the effect of fluazifop rate on injury ratings of each of the four tested genotypes at 21 DAT. The two-way analysis of variance indicated significant genotype, herbicide rate, and genotype by herbicide rate effects for injury ratings at 21, and 28 DAT (data not shown). Both Line A and Line B showed significantly less injury than PT and TCC at all rates above 50 g ai ha⁻¹. Estimates of LD₅₀ for the four genotypes were 36, 37, 800, and 516 g ai ha⁻¹ for PT, TC, Line A, and Line B, respectively. These data provide strong evidence of the presence of cross resistance to fluazifop in both Line A and Line B. The level of cross resistance present is adequate to provide effective control of susceptible weedy grasses without serious concerns over herbicide injury.

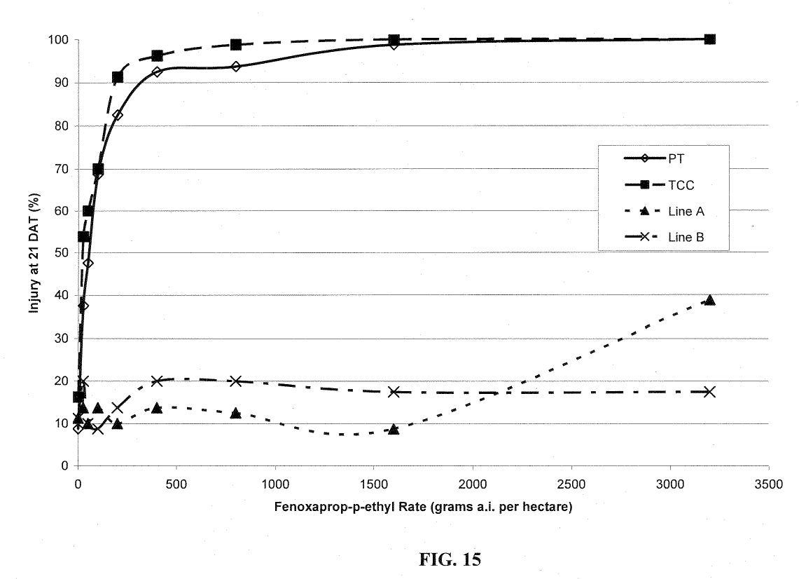

FIG. 15 illustrates the effect of fenoxaprop rate on injury ratings of each of the four tested genotypes at 21 DAT. The two-way analysis of variance indicated significant genotype, herbicide rate, and genotype by herbicide rate effects for injury ratings at 21, and 28 DAT (data not shown). Both Line A and Line B showed significantly less injury than PT and TCC at all rates above 50 g ai ha⁻¹. In this experiment both Line A and Line B expressed very high levels of cross resistance to fenoxaprop. Line A was injured less than 20% at all fenoxaprop rates up 1600 g ai ha⁻¹ and Line B was injured less than 20% even at the highest rate of 3200 g ai ha⁻¹. Estimates of LD₅₀ for the four genotypes were 56, 22, >3200, and >3200 g ai ha⁻¹ for PT, TC, Line A, and Line B, respectively. These data provide strong evidence of the presence of cross resistance to fenoxaprop in both Line A and Line B. The level of cross resistance present is more than adequate to provide effective control of susceptible weedy grasses without serious concerns over herbicide injury.

### Example 11

### Selection of Sethoxydim-Resistant Cell Lines in Bent Grass

To induce callus tissue formation, seeds of bent grass are surface-sterilized in 10% bleach for four hours while being vigorously shaken. The sterilized seeds are then placed on callus induction medium as described in Table 5 (Luo, et al. 2003. Plant Cell Reports 22(9):645-652, which is incorporated herein by reference in its entirety).

TABLE 5
Callus induction medium for bent grass

| Component | Concentration (per liter of medium) |
|---|---|
| MS/B5 medium | (Murashige and Skoog. 1962. supra; Gamborg |
| | et al. 1968. supra) |
| Dicamba | 6.6 mg |
| Casein hydrolysate | 500 mg |
| Sucrose | 30 g |
| Gelrite ® | 2 g |

Once callus tissue from bent grass is obtained, the calli are screened by the sethoxydim selection process as previously described (Example 4). Briefly, selection of sethoxydim resistant (SR) cells is performed by placing callus tissue on callus induction medium (Table 5) containing 10 μM sethoxydim. Large plates (245×245 mm in size) are used to efficiently screen greater numbers of cells. Callus tissue approximately 4-mm in diameter are placed in a 15×15 grid, giving a total of 225 calli per plate. Calli are subcultured three times at three-week intervals (Example 3) for a total selection period of nine weeks. Resistant calli are subcultured into 100×15 mm petri dishes containing callus induction medium (Table 5) supplemented with 10 μM sethoxydim for one month in order to obtain sufficient callus. This provided a total selection time of 12 weeks or more.

### Example 12

### Regeneration of Sethoxydim-Resistant Cell Lines in Bent Grass

Once sethoxydim-resistant calli are obtained, regeneration is attempted on all resistant calli. The regeneration medium used as as described in Table 6 (Luo, et al. 2003. supra).

TABLE 6
Regeneration medium for bent grass

| Component | Concentration (per liter of medium) |
|---|---|
| MS/B5 medium | (Murashige and Skoog. 1962. supra; |
| | Gamborg et al. 1968. supra) |
| Myo-inositol | 100 mg |
| 6-benzylaminopurine (BAP) | 1 mg |
| Sucrose | 30 g |
| Gelrite ® | 2 g |

Any regeneration protocol known to those of skill in the art can be conducted for regeneration of sethoxydim-resistant bent grass calli. An exemplary regeneration protocol is described in Luo, et al. (2003. supra), Another exemplary regeneration protocol is described in Example 5.

### Example 13

### Molecular Characterization of Sethoxydim Resistant Lines in Bent Grass

Once sethoxydim-resistant (SR) bent grass lines are identified, the mutation causing the resistance can be characterized. An exemplary protocol to identify a mutation at position 1781 of the ACCase gene is describe herein (Example 6). In addition, the bent grass lines can be analyzed for mutations at any other positions in the ACCase gene by designing primers to amplify specific regions that include positions 2027, 2041, 2078 (Example 6) and 2096 (Délye. 2005. supra). Designing primers and amplifying regions for sequence analysis is well known to those of skill in the art.

### Example 14

### Evaluation of Whole Plant Resistance to Sethoxydim and ACCase Inhibitors Herbicides in Bent Grass

Once sethoxydim-resistant bent grass plants are regenerated, whole plant resistance to sethoxydim can be conducted as herein described (Examples 8 and 9). In addition, cross-resistance to other ACCase inhibitor herbicides can be carried out as herein described (Example 10),

### Example 15

### Induction of Callus Tissue from Tall Fescue Grass

To induce callus tissue formation, seeds of tall fescue grass are sterilized in 50% sulfuric acid for 30 minutes, rinsed with deionized water and 95% ethanol, and stirred in 100% bleach with 0.1% tween for 30 minutes. The seeds are then rinsed in sterile water 10 times for four minutes each time. Once sterilized, the seeds are placed on MS/B5D2 medium (Murashige and Skoog. 1962. supra; Gamborg et al. 1968. supra) for germination. One week later, all germinated seeds are injured by slicing the seeds to promote callus growth. The sliced seeds are placed in a callus induction medium as described in Table 7 to induce formation of callus tissue. The calli are transferred every two weeks for propagation for use in further experiments.

TABLE 7
Callus induction medium for tall fescue grass

| Component | Concentration (per liter of medium) |
|---|---|
| MS basal salts | (Murashige and Skoog. 1962. supra) |
| B5 vitamins | (Gamborg et al. 1968. supra) |
| Sucrose | 30 mg |
| 2,4-D | 5 mg |
| 6-benzylaminopurine (BAP) | 0.15 mg |
| Gelzan ™ | 3 g |

### Example 16 Selection of Sethoxydim-Resistant Cell Lines in Tall Fescue Grass

Once callus tissue from tall fescue grass is obtained, the calli can be screened by the sethoxydim selection process as previously described (Example 4). Briefly, selection of sethoxydim resistant (SR) cells is performed by placing callus tissue on callus induction medium (Table 7) containing 10 μM sethoxydim. Large plates (245×245 mm in size) are used to efficiently screen greater numbers of cells. Callus tissue approximately 4-mm in diameter is placed in a 15×15 grid, giving a total of between about 200 to 250 calli per plate. Calli are subcultured three times at two-week intervals (Example 3). Resistant calli are subcultured into 100×15 mm petri dishes containing callus induction medium (Table 7) supplemented with 10 μM sethoxydim and propagated for at least one month in order to obtain sufficient callus.

### Example 17

### Regeneration of Sethoxydim-Resistant Cell Lines in Tall Fescue Grass

Once sethoxydim-resistant calli are obtained, regeneration is attempted on all resistant calli. An exemplary regeneration medium as described in Table 6 (Luo, et al. 2003. supra) can be used. Another exemplary regeneration protocol is described in Example 5. However, any regeneration protocol known to those of skill in the art can be conducted for regeneration of sethoxydim-resistant tall fescue calli.

### Example 18

### Molecular Characterization of Sethoxydim Resistant Lines in Tall Fescue Grass

Once sethoxydim-resistant (SR) tall fescue lines are identified, the mutation causing the resistance can be characterized. An exemplary protocol to identify a mutation at position 1781 of the ACCase gene is describe herein (Example 6). In addition, the tall fescue lines can be analyzed for mutations at any other positions in the ACCase gene by designing primers to amplify specific regions that include positions 2027, 2041, 2078 (Example 6) and 2096 (Délye. 2005. supra). Designing primers and amplifying regions for sequence analysis is well known to those of skill in the art.

### Example 19

### Evaluation of Whole Plant Resistance to Sethoxydim and ACCase Inhibitors Herbicides in Tall Fescue

Once sethoxydim-resistant tall fescue plants are regenerated, whole plant resistance to sethoxydim can be conducted as herein described (Examples 8 and 9). In addition, cross-resistance to other ACCase inhibitor herbicides can be carried out as herein described (Example 10),

### Example 20

### Selection of Sethoxydim-Resistant Cell Lines in Zoysiagrass

To induce callus tissue formation, seeds of zoysiagrass are sterilized in 50% sulfuric acid for 30 minutes, rinsed with deionized water and 95% ethanol, and stirred in 100% bleach with 0.1% tween for 30 minutes. The seeds are then rinsed in sterile water 10 times for four minutes each time. Once sterilized, the seeds are placed on MS/B5D2 medium (Murashige and Skoog. 1962. supra; Gamborg et al. 1968. supra) for germination. One week later, all germinated seeds are injured by slicing the seeds to promote callus growth. The sliced seeds are placed in a callus induction medium as described in Table 7 to induce formation of callus tissue. The calli are transferred every two weeks for propagation for use in further experiments.

### Example 21

### Selection of Sethoxydim-Resistant Cell Lines in Zoysiagrass

Once callus tissue from zoysiagrass is obtained, the calli can be screened by the sethoxydim selection process as previously described (Example 4). Briefly, selection of sethoxydim resistant (SR) cells is performed by placing callus tissue on callus induction medium (Table 7) containing 10 μM sethoxydim. Large plates (245×245 mm in size) are used to efficiently screen greater numbers of cells. Callus tissue approximately 4-mm in diameter is placed in a 15×15 grid, giving a total of between about 200 to 250 calli per plate. Calli are subcultured three times at two-week intervals (Example 3). Resistant calli are subcultured into 100×15 mm petri dishes containing callus induction medium (Table 7) supplemented with 10 μM sethoxydim and propagated for at least one month in order to obtain sufficient callus.

### Example 22

### Regeneration of Sethoxydim-Resistant Cell Lines in Zoysiagrass

Once sethoxydim-resistant calli are obtained, regeneration is attempted on all resistant calli. An exemplary regeneration medium as described in Table 6 (Luo, et al. 2003. supra) can be used. Another exemplary regeneration protocol is described in Example 5. However, any regeneration protocol known to those of skill in the art can be conducted for regeneration of sethoxydim-resistant zoysiagrass calli.

### Example 23

### Molecular Characterization of Sethoxydim Resistant Lines in Tall Fescue Grass

Once sethoxydim-resistant (SR) tall fescue lines are identified, the mutation causing the resistance can be characterized. An exemplary protocol to identify a mutation at position 1781 of the ACCase gene is describe herein (Example 6). In addition, the tall fescue lines can be analyzed for mutations at any other positions in the ACCase gene by designing primers to amplify specific regions that include positions 2027, 2041, 2078 (Example 6) and 2096 (Délye. 2005. supra). Designing primers and amplifying regions for sequence analysis is well known to those of skill in the art.

### Example 24

### Evaluation of Whole Plant Resistance to Sethoxydim and ACCase Inhibitors Herbicides in Zoysiagrass

Once sethoxydim-resistant zoysiagrass plants are regenerated, whole plant resistance to sethoxydim can be conducted as herein described (Examples 8 and 9). In addition, cross-resistance to other ACCase inhibitor herbicides can be carried out as herein described (Example 10),

### Example 25

### Controlling Weedy Species Among Herbicide-Resistant Plants by Application of an Herbicide

A plot containing both bermudagrass and sethoxydim-resistant seashore paspalum is treated with 150 g a.i. ha⁻¹ sethoxydim once a week over a period of three months. Over the three month treatment period, it is observed that the bermudagrass slowly dies out while the sethoxydim-resistant paspalum continues to thrive, leaving the plot populated with above 80% sethoxydim-resistant paspalum.

### Example 26

### Controlling Weedy Species Among Herbicide-Resistant Plants by Application of a Combination Herbicide

A plot containing both bermudagrass and sethoxydim-resistant seashore paspalum is treated with both 150 g a.i. ha⁻¹ sethoxydim and 150 g a.i. ha⁻¹ fenoxaprop once a week over a period of three months. Over the three month treatment period, it is observed that the bermudagrass slowly dies out while the sethoxydim-resistant paspalum continues to thrive, leaving the plot populated with above 80% sethoxydim-resistant paspalum.

### Example 27

### Marker-Assisted Selection: Identifying Traits Suitable for Selection Using Herbicide Resistance as a Marker

A tall fescue variety having several traits desirable for breeding purposes is cultured as discussed herein (see Examples 15-19) to identify sethoxydim-resistant callus lines of the variety. These lines are regenerated to mature plants of generation R₀. R₀ plants having the ACCase I1781L mutation, conferring sethoxydim resistance, are crossed with a different tall fescue variety lacking the several traits. Through subsequent crosses, certain of the desirable traits are shown to segregate non-randomly with sethoxydim resistance. Through further optional crosses, linkage between sethoxydim resistance and each of the linked traits can be quantified. For each trait found to be linked to sethoxydim resistance, such resistance is a useful marker for marker-assisted breeding/selection protocols.

### Example 28

### Marker-Assisted Selection: Selecting a Desirable Linked Trait Based Upon Marker Phenotype

Sethoxydim-resistant tall fescue plants from Example 27, of the R₀ generation or progeny of such generation, are used for marker-assisted breeding and selection. A commercial variety of tall fescue lacking one of the linked traits identified in Example 27 is crossed with the sethoxydim-resistant tall fescue plants from Example 27 to form a hybrid generation. Seeds of the hybrid generation are germinated and the plants are treated with sethoxydim at a level sufficient to kill or severely retard the growth of non-resistant plants. Healthy, sethoxydim-resistant plants are selected for further crosses. A large proportion of such selected plants carry the linked trait. Further generations of crosses between sethoxydim-resistant plants with plants of the commercial variety, followed by sethoxydim treatment and selection, result in a plant line having substantially the genetic background of the commercial variety, but carrying the desirable trait that was confirmed to be linked to sethoxydim resistance.

### Example 29

### Marker-Assisted Selection: Selecting a Desirable Linked Trait Based Upon a Molecular Marker

Sethoxydim-resistant tall fescue plants from Example 27, of the R₀ generation or progeny of such generation, are used for marker-assisted breeding and selection. A commercial variety of tall fescue lacking one of the linked traits indentified in Example 27 is crossed with the sethoxydim-resistant tall fescue plants from Example 27 to form a hybrid generation. Seeds of the hybrid generation are germinated and samples from the germinated plants are screened by molecular methods such as PCR for presence of the SNP associated with the I1781L mutation. For example, the SV384F and SV384R primers (Example 6, SEQ ID NOs: 1 and 2) can be used in an amplification assay to detect the marker. Presence of the molecular marker in a hybrid plant confirms a likelihood that the hybrid plant also carries the desirable traits linked to sethoxydim resistance, as discussed in Example 27. Plants carrying the molecular marker are selected for further crosses. A large proportion of such selected plants carry the linked trait. Further generations of crosses between plants having the marker, with plants of the commercial variety, followed by either further molecular selection or by sethoxydim treatment and selection, result in a plant line having substantially the genetic background of the commercial variety, but carrying the desirable trait that was confirmed to be linked to sethoxydim resistance.

The various methods and techniques described above provide a number of ways to carry out the invention. Furthermore, the skilled artisan will recognize the applicability of various features from different embodiments. Similarly, the various elements, features and steps discussed above, as well as other known equivalents for each such element, feature or step, can be combined and/or modified by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

What is claimed is:

1. A selected and cultured ACCase inhibitor herbicide-resistant plant from the group Panicodae, or tissue, seed, or progeny thereof.

2. The ACCase inhibitor herbicide-resistant plant of claim 1, regenerated from an herbicide-resistant undifferentiated cell that has undergone a selection method, wherein the selection method comprises:
providing a callus of undifferentiated cells of a plant from the group Panicodae;
contacting the callus with at least one herbicide in an amount sufficient to retard growth or kill the callus;
selecting at least one resistant cell based upon a differential effect of the herbicide; and
regenerating a viable whole plant of the variety from the at least one resistant cell.

3. The ACCase inhibitor herbicide-resistant plant of claim 1, wherein the plant is a member of tribe Paniceae.

4. The ACCase inhibitor herbicide-resistant plant of claim 3, wherein the plant is one selected from the group of: Axonopus (carpetgrass), Digiteria (crabgrass), Echinochloa, Panicum, Paspalum (Bahiagrass), Pennisetum, Setaria and Stenotaphrum (St. Augustine grass).

5. The ACCase inhibitor herbicide-resistant plant of claim 3, wherein the plant is one selected from the group of: seashore paspalum (P. vaginatum), bent grass, tall fescue grass, Zoysiagrass, bermudagrass (Cynodon spp), Kentucky Bluegrass, Texas Bluegrass, Perennial ryegrass, buffalograss (Buchloe dactyloides), centipedegrass (Eremochloa ophiuroides) and St. Augustine grass (Stenotaphrum secundatum), Carpetgrass (Axonopus spp.) and Bahiagrass (Paspalum notatum).

6. The ACCase inhibitor herbicide-resistant plant of claim 1, wherein the plant is resistant to an acetyl coenzyme A carboxylase (ACCase) inhibitor.

7. The ACCase inhibitor herbicide-resistant plant of claim 1, wherein the plant is resistant to a cyclohexanedione herbicide, an aryloxyphenoxy proprionate herbicide, a phenylpyrazoline herbicide, or mixtures thereof.

8. The ACCase inhibitor herbicide-resistant plant of claim 1, wherein the herbicide resistance is conferred by a mutation at least one amino acid position of ACCase gene selected from the group of: 1756, 1781, 1999, 2027, 2041, 2078, 2099 and 2096.

9. The ACCase inhibitor herbicide-resistant plant of claim 8, wherein the herbicide resistance is conferred by an isoleucine to leucine mutation at amino acid position 1781

10. The ACCase inhibitor herbicide-resistant plant of claim 1, wherein the plant is resistant to at least one herbicide selected from the group of: alloxydim, butroxydim, cloproxydim, profoxydim, sethoxydim, clefoxydim, clethodim, cycloxydim, tepraloxydim, tralkoxydim, chloraizfop, clodinafop, clofop, cyhalofop, diclofop, fenoxaprop, fenthiaprop, fluazafop-butyl, fluazifop, haloxyfop, isoxapyrifop, metamifop, propaquizafop, quizalofop, trifop and pinoxaden.

11. The ACCase inhibitor herbicide-resistant plant of claim 1, wherein the plant is a non-transgenic plant.

12. A progeny of an ACCase inhibitor herbicide-resistant plant of claim 1.

13. The progeny of claim 12, wherein the progeny is a result of sexual reproduction of the ACCase inhibitor herbicide-resistant plant parent.

14. The progeny of claim 12, wherein the progeny is a result of asexual reproduction of the ACCase inhibitor herbicide-resistant plant parent.

15. A seed of an ACCase inhibitor herbicide-resistant plant of claim 1, or a progeny thereof.

16. A method of identifying a herbicide-resistant plant from the group Panicodae, comprising:
providing a callus of undifferentiated cells of a plant from the group Panicodae;
contacting the callus with at least one herbicide in an amount sufficient to retard growth or kill the callus;
selecting at least one resistant cell based upon a differential effect of the herbicide; and
regenerating a viable whole plant of the variety from the at least one resistant cell,
wherein the regenerated plant is resistant to the at least one herbicide.

17. The method of claim 16, further comprising expanding the at least one resistant cell into a plurality of undifferentiated cells.

18. The method of claim 16, wherein the plant is one selected from the tribe Paniceae.

19. The method of claim 18, wherein the plant is one selected from the group of: Axonopus (carpetgrass), Digiteria (crabgrass), Echinochloa, Panicum, Paspalum (Bahiagrass), Pennisetum, Setaria and Stenotaphrum (St. Augustine grass).

20. The method of claim 18, wherein the plant is one selected from the group of:
seashore paspalum (P. vaginatum), bentgrass (Agrostis spp), tall fescue, Zoysiagrass, bermudagrass (Cynodon spp), Kentucky Bluegrass, Texas Bluegrass, Perennial ryegrass, buffalograss (Buchloe dactyloides), centipedegrass (Eremochloa ophiuroides) and St. Augustine grass (Stenotaphrum secundatum), Carpetgrass (Axonopus spp.) and Bahiagrass (Paspalum notatum).

21. The method of claim 16, wherein the at least one herbicide is an acetyl coenzyme A carboxylase (ACCase) inhibitor.

22. The method of claim 21, wherein the herbicide resistance is conferred by a mutation at least one amino acid position of the ACCase gene selected from the group of: 1756, 1781, 1999, 2027, 2041, 2078, 2099 and 2096.

23. The method of claim 22, wherein the herbicide resistance is conferred by an isoleucine to leucine mutation at amino acid position 1781.

24. The method of claim 21, wherein the at least one herbicide is selected from the group of: alloxydim, butroxydim, cloproxydim, profoxydim, sethoxydim, clefoxydim, clethodim, cycloxydim, tepraloxydim, tralkoxydim, chloraizfop, clodinafop, clofop, cyhalofop, diclofop, fenoxaprop, fenthiaprop, fluazafop-butyl, fluazifop, haloxyfop, isoxapyrifop, metamifop, propaquizafop, quizalofop, trifop and pinoxaden.

25. The method of claim 16, wherein the callus of undifferentiated cells is provided from a non-transgenic plant.

26. A tissue culture of regenerable cells of an herbicide-resistant plant identified by the method of claim 16.

27. A method for controlling weeds in the vicinity of a herbicide-resistant plant identified by the method of claim 16, comprising: contacting at least one herbicide to the weeds and to the herbicide-resistant plant, wherein the at least one herbicide is contacted to the weeds and to the plant at a rate sufficient to inhibit growth of a non-selected plant of the same species or sufficient to inhibit growth of the weeds.

28. The method of claim 27, wherein the herbicide-resistant plant is resistant to an acetyl coenzyme A carboxylase (ACCase) inhibitor.

29. The method of claim 27, wherein the herbicide-resistant plant is a non-transgenic plant.

30. The method of claim 27, comprising contacting the herbicide directly to the herbicide-resistant plant.

31. The method of claim 27, comprising contacting the herbicide to a growth medium in which the herbicide-resistant plant is located.

32. A seashore paspalum-specific DNA marker deposited as ATCC Deposit No. ______, or a fragment thereof, that is capable of identifying herbicide-resistant grass cultivars.

33. A method of marker-assisted breeding, comprising the steps of:
identifying a feature of interest for breeding and selection, wherein the feature is in linkage with an ACCase gene;
providing a first plant carrying an ACCase sequence variant capable of conferring upon the plant resistance to an ACCase-inhibitor herbicide, wherein the plant further comprises the feature of interest;
breeding the first plant with a second plant;
identifying progeny of the breeding step as having the ACCase sequence variant; and
selecting progeny likely to have the feature of interest based upon the identifying step.

34. The method of claim 33, wherein the feature is selected from: a trait or, a gene.

35. The method of claim 34, wherein the trait is at least one selected from the group consisting of: herbicide tolerance, disease resistance, insect of pest resistance, altered fatty acid, protein or carbohydrate metabolism, increased growth rates, enhanced stress tolerance, preferred maturity, enhanced organoleptic properties, altered morphological characteristics, sterility, other agronomic traits, traits for industrial uses, or traits for improved consumer appeal.
